(19) United States
(12) Patent Application Publication
(10) Pub. No.: US 2023/0165514 A1
(43) Pub. Date: Jun. 1, 2023

(54) SYSTEMS AND METHODS FOR ADMINISTERING A MOTOR ASSESSMENT TO SCREEN FOR EARLY MILD COGNITIVE IMPAIRMENT (MCI) OR OTHER COGNITIVE AND/OR NEUROLOGICAL CONCERNS

(71) Applicant: Arizona Board of Regents on Behalf of Arizona State University, Tempe, AZ (US)

(72) Inventor: Sydney Schaefer, Tempe, AZ (US)

(73) Assignee: Arizona Board of Regents on Behalf of Arizona State University, Tempe, AZ (US)

(21) Appl. No.: 18/060,059

(22) Filed: Nov. 30, 2022

Related U.S. Application Data
(60) Provisional application No. 63/378,168, filed on Oct. 3, 2022.
(60) Provisional application No. 63/284,580, filed on Nov. 30, 2021.

(51) Int. Cl. A61B 5/00; A61B 5/11; A61B 5/0531

(52) U.S. Cl. CPC A61B 5/4088; A61B 5/1124; A61B 5/0531

(57) ABSTRACT

A system includes a plurality of test elements and a processor. The test elements are configured for execution of a motor task by an individual and generation of test data from trials of the motor task. The processor is configured to compute a motor test score from the test data to assess a potential neurological concern.

### CROSS REFERENCE TO RELATED APPLICATIONS

The present application claims the benefit of U.S. Provisional Patent Application No. 63/284,580 filed on Nov. 30, 2021 and entitled “SYSTEMS AND METHODS FOR DIAGNOSIS AND PROGNOSIS OF COGNITIVE IMPAIRMENT”, and further claims the benefit of U.S. Provisional Patent Application No. 63/378,168 filed on Oct. 3, 2022 and entitled “SYSTEMS AND METHODS FOR ADMINISTERING A MOTOR ASSESSMENT TO SCREEN FOR EARLY MILD COGNITIVE IMPAIRMENT (MCI) OR OTHER COGNITIVE AND/OR NEUROLOGICAL CONCERNS”; all of which is hereby incorporated by reference in their entireties.

### FEDERALLY SPONSORED RESEARCH OR DEVELOPMENT

The present invention was made with government support under K01 AG047926 and R03 AG056822 awarded by the National Institutes of Health. The government has certain rights in the invention.

### FIELD

The present disclosure generally relates to motor assessments and cognitive function; and more particularly, to systems and methods for computer-implemented motor assessments to screen for mild cognitive impairment (MCI) or any neurological condition as described herein.

### BACKGROUND

A visit to a primary care physician's office typically involves some initial examination of a patient prior to the patient engaging with the physician. For example, the patient is often weighed and measured with respect to height, and one or more of the patient's vital signs may be analyzed (e.g., blood pressure, temperature, and the like). These vital signs are commonly taken to assess the general health of the patient prior to examination, and/or to identify possible issues. However, cognitive function of the patient may be overlooked, and may be a contributing factor to vital signs, or a useful indicator that neuropsychological examination is needed. Presently, technology is lacking with respect to functional motor assessment at the physician's office or any remote location, and a need exists for technical improvements to accommodate portable and/or efficient motor assessments.

It is with these observations in mind, among others, that various aspects of the present disclosure were conceived and developed.

### SUMMARY

Aspects of the present inventive concept may take the form of a system, comprising a plurality of test elements and a processor configured to compute a motor score based on data associated with an individual's engagement with the plurality of test elements. The plurality of testing components is configured for interaction with an individual to conduct one or more motor assessments, the one or more motor assessments including engagement of upper extremities of the individual to acquire at least one object of the plurality of testing components from a source location and transport the one or more objects to a target location. The processor is configured to access a plurality of trial times and test scores associated with the one or more motor assessments, and compute a motor score, the motor score reflecting a potential concern for a neurological condition based upon a predefined score threshold.

In some embodiments, the system further includes a user interface executed by a computing device, the computing device configured to provide, via the user interface, a stopwatch function and a scoring function that the individual engages to accommodate aggregation of the plurality of trial times and test scores for access by the processor.

In some embodiments, the one or more motor assessments includes a plurality of goal directed movements to visible target locations, spatially arranged such that at least one target is located ipsilateral to the reaching extremity, at least one target is located contralateral to the reaching extremity, and one target is located along the individual's midline.

In some embodiments, the one or more motor assessments includes a sequence of target locations to indicate the order in which the individual must transport the one or more objects, the sequence of target locations being the same across a plurality of trials of the one or more motor assessments.

In some embodiments, the system includes a virtual reality subsystem in operable communication with the processor, including: a virtual reality (VR) device in operable communication with the processor, the VR device configured to generate a simulated environment and configured for interaction with the individual to conduct the one or more motor assessments via the simulated environment, wherein the plurality of testing components includes features of the simulated environment rendered by the VR device. In some embodiments, the virtual reality subsystem further comprises: a VR controller in operable communication with the VR device that accommodates simulated movement of the individual throughout the simulated environment and interaction with a simulated object to conduct the one or more motor assessments. Aspects of the present inventive concept may further take the form of a method, comprising the steps of: providing a plurality of test elements, the plurality of test elements configured for interaction with an individual to execute a plurality of trials of a motor task according to a predetermined sequence, the motor task including engagement of upper extremities of the individual to acquire a first object of the plurality of test elements from a source location and transport the first object to a first target location; and executing, by a processor, steps of: accessing test data including a first plurality of trial times associated with the plurality of trials of the motor task, and computing a motor test score, the motor score reflecting a potential concern for a neurological condition based upon a predefined score threshold.

Aspects of the present inventive concept may further take the form of a kit, comprising a container configured for storage and transportation of a plurality of test elements and an instruction. In some examples, the kit includes a container; a plurality of testing elements including one or more objects and a plurality of receptacles, the container configured for secure storage and transport of the plurality of testing elements; and an instruction for guiding an individual to complete one or more motor tasks using the plurality of testing elements according to a predetermined sequence configured for detecting a neurological concern.

Another aspect of the present inventive concept includes a tool for use by a subject in a motor task. In various embodiments, the tool comprises a handle and a repository, wherein the repository is configured to receive and hold at least one object; wherein the handle comprises at least one sensor configured to collect data and a timer. In embodiments, the at least one sensor comprises a pressure sensor, a skin conductance sensor, or a combination thereof. The pressure sensor can be configured to measure changes in a grip force during the motor task. In embodiments, the skin conductance sensor is configured to measure electrodermal response due to physiological arousal.

In certain embodiments, the at least one sensor is configured to transmit the data to an application running on a processor of a mobile computing device. The application can be configured to compare the data with aggregate patient data.

Another aspect of the invention comprises a system for diagnosis or prognosis of a neurological condition in a subject. In embodiments, the system comprises a tool configured to receive, hold, and manipulate at least one object during a motor task, wherein the tool comprises a timer and at least one tool sensor. The system can further comprise a home receptacle configured to receive and hold the at least one object and a target receptacle configured to receive and hold at least one object. In embodiments, the system the at least one sensor comprises a pressure sensor, a skin conductance sensor, or a combination thereof wherein the pressure sensor is configured to measure changes in a grip force during the motor task and wherein the skin conductance sensor is configured to measure electrodermal response due to physiological arousal. The system can also include a support board configured to support the home receptacle and target receptacle thereon. The support board can comprise an optical hand tracking module configured to record bodily movements during the motor task. In certain embodiments, the system comprises an eye tracker configured to measure pupil dilation throughout the motor task. In certain embodiments, a bottom surface of the home receptacle, the target receptacle, or both comprises an object pressure sensor that is configured to detect the presence or absence of at least one object during the motor task.

In various embodiments, the neurological condition comprises Alzheimer's disease, behavioral variant frontotemporal dementia, corticobasal degeneration, Huntington's disease, Lewy body dementia, mild cognitive impairment, primary progressive aphasia, progressive supranuclear palsy, vascular dementia, Parkinson's disease, William's syndrome, autism, or a history of stroke.

Another aspect of the present inventive concept includes a noninvasive method of predicting hippocampal volume of a subject. In embodiments, the method comprises subjecting the subject to a motor task comprising a plurality of trials, wherein, during each trial, the subject employs a tool as described in any of the various exemplary embodiments disclosed herein to acquire and transport one or more objects at a time from a home receptacle to a plurality of target receptacles and obtaining a task assessment score wherein the task assessment score comprise the variability of time required to complete each trial. In embodiments, a high degree of variability indicates that the subject has a reduced hippocampal volume.

Another aspect of the present inventive concept is a noninvasive method of assessing cortical amyloid deposition a subject. In embodiments, the method comprises subjecting the subject to a motor task comprising a plurality of trials, wherein, during each trial, the subject employs a tool as described in any of the various exemplary embodiments disclosed herein to acquire and transport one or more objects at a time from a home receptacle to a plurality of target receptacles and obtaining a task assessment score wherein the task assessment score comprise the variability of time associated with completion or execution of each trial. In embodiments, a high degree of variability indicates that the subject has a high degree of cortical amyloid deposition.

Yet another aspect includes a method of pre-screening a subject for a clinical trial. In embodiments, the method comprises subjecting the subject to a motor task comprising a plurality of trials, wherein, during each trial, the subject employs a tool as described in any of the various exemplary embodiments disclosed herein to acquire and transport one or more objects from a home receptacle to a plurality of target receptacles and obtaining a task assessment score wherein the task assessment score comprise the variability of time required to complete each trial. In embodiments, a high degree of variability indicates that the subject should be admitted to the clinical trial.

Another aspect of the present inventive concept includes a method of diagnosing a neurological condition. In embodiments, the method comprises subjecting the subject to a motor task comprising a plurality of trials, wherein, during each trial, the subject employs a tool as described in any of the various exemplary embodiments disclosed herein to acquire and transport one or more objects at a time from a home receptacle to a plurality of target receptacles. The method can further comprise obtaining a task assessment score wherein the task assessment score comprise the variability of time required to complete each trial and diagnosing the subject with the neurological condition if the task assessment score comprises a high degree of variability.

An additional aspect comprises a method of determining a therapeutic efficacy of a drug in treating a neurological condition. In certain embodiments, the method comprises subjecting a subject diagnosed with the neurological condition to a first motor task comprising a plurality of trials, wherein, during each trial, the subject employs a tool as described in any of the various exemplary embodiments disclosed herein to acquire and transport one or more objects at a time from a home receptacle to a plurality of target receptacles. The method further comprises obtaining a first task assessment score wherein the task assessment score comprises the variability of time required to complete each trial of the first motor task. The method can also include administering the drug during a trial treatment period. After the trial treatment period, the method includes subjecting the subject to a second motor task, wherein the second motor task comprises the same steps as the first motor task and obtaining a second task assessment score, wherein the second task assessment score comprises the variability of time required to complete the second motor task. In embodiments, the method further comprises determining that the drug is therapeutically efficacious if the second task assessment score is improved compared to that of the first task assessment score.

Another aspect of the inventive concept includes a method of determining the progression of a neurological condition in a subject. In various embodiments, the method comprises subjecting the subject to a first motor task comprising a plurality of trials, wherein, during each trial, the subject employs a tool as described in any of the various exemplary embodiments disclosed herein to acquire and transport one or more objects at a time from a home receptacle to a plurality of target receptacles. The method can further comprise obtaining a first task assessment score wherein the task assessment score comprises the variability of time required to complete each trial of the first motor task. In embodiments, the method comprises the step of permitting an assessment time period to pass, subjecting the subject to a second motor task, and obtaining a second motor task assessment score following the assessment period, wherein the second motor task comprises the same steps as the first motor task, and determining progression of the neurological condition.

In various embodiments, of the methods described herein, the neurological condition comprises Alzheimer's disease, behavioral variant frontotemporal dementia, corticobasal degeneration, Huntington's disease, Lewy body dementia, mild cognitive impairment, primary progressive aphasia, progressive supranuclear palsy, vascular dementia, Parkinson's disease, William's syndrome, autism, or a history of stroke.

In various embodiments of the methods described herein, the motor task comprises three target receptacles. Each of the three target receptacles can be positioned along a radius surrounding the home target such that each of the three target receptacles are equidistant from the home target. In one embodiment, the first receptacle is placed at −40° along the radius in relation to the home receptacle, the second receptacle is placed at 0° along the radius in relation to the home receptacle, and the third receptacle is placed at 40° along the radius in relation to the home receptacle.

In certain embodiments of the methods disclosed herein, the task assessment score comprises the time required to complete one or more trials of the motor task, changes in motor task performance from across two or more trials, the time required to remove an object and from the home receptacle and deposit the object in the target receptacle, the pressure applied by a subject when holding or manipulating the tool, the number of grip changes during a trial, the number of movement errors during a trial, the number of times an object is dropped during a trial, the angle of the tool during a trial, skin conductance during a trial, the position of the subject's hands, or a combination thereof.

The motor task can comprise at least six trials. In embodiments, the motor task comprises up to fifteen trials.

In certain embodiments described herein, the subject transports two objects at a time.

The task can include a functional upper-extremity assessment using adaptive fine motor skill. The motor task can comprise a timed assessment of upper-extremity functionality. In embodiments, the motor task assesses functional upper-extremity movement in a subject wherein the subject employs a tool to acquire and transport one or more objects from one receptacle (or container) to another. The motor task can comprise one or more trials, wherein each trial comprises acquiring and transporting one or more objects at a time with a tool from a ‘home’ receptacle to one or more “target” receptacle. Embodiments can comprise one or more practice trials followed by one or more performance trials, wherein a task assessment score is obtained during the one or more performance trials.

Other objects and advantages of this inventive concept will become readily apparent from the ensuing description.

### BRIEF DESCRIPTION OF THE DRAWINGS

FIG. 1A is a simplified block diagram of a general non-limiting computer-implemented system for detection of a cognitive concern or variability.

FIG. 1B is an illustration of an example kit for implementing aspects of the inventive concept described herein.

FIG. 1C is an illustration of an example motor task and generation of test data from an individual executing one or more trials of the motor task.

FIG. 1D is a simplified illustration of an example arrangement of the plurality of test elements for a motor task.

FIG. 1E is an example demonstration of a sequence associated with the motor task of FIG. 1D.

FIG. 1F is an example method associated with the system and other aspects of the present disclosure described in FIGS. 1A-1E.

FIG. 2A is a graph showing a graphical relationship between age and a motor task performance according to an example associated with the present inventive concept.

FIG. 2B is a graph illustrating estimated trail time of the non-dominant hand across four practice trials calculated from a linear mixed-effects model under one non-limiting embodiment.

FIG. 2C is an illustration of a map of the United States showing data reporting details associated with FIGS. 2A-2B.

FIG. 3 is a graph illustrating task performance across six practice trails for a cognitively impaired participant.

FIG. 4A is a graph illustrating receiver operator characteristics for predicting the probability of amyloid positivity.

FIG. 4B is a graph illustrating precision recall curves for predicting the probability of amyloid positivity.

FIG. 5A is an illustration showing task acquisition and amyloid burden from a first subject.

FIG. 5B is an illustration showing task acquisition and amyloid burden from a second subject.

FIG. 6A is a graph illustrating trial-by-task performance for cognitively intact, MCI, and mild AD samples in APPE baseline.

FIG. 6B is a graph illustrating a performance curve associated with FIG. 6A.

FIG. 7A is a graph illustrating mean (SE) task performance for cognitively intact participants in APPE (n=16), by APOE genotype.

FIG. 7B is a graph illustrating corresponding performance curves associated with FIG. 7A for cognitively intact participants in APPE (n=16), by APOE genotype.

FIG. 8 is a graph of performance curves for cognitively-intact, MCI, and mild AD samples in APPE at one-year follow-up.

FIG. 9A is a graph illustrating task performance being more variable during acquisition for the AD conversion group described herein as compared with the stable group particularly after a first trial.

FIG. 9B is a graph showing mean (SE) within-subject variability being higher for the AD conversion group (p=0.07).

FIG. 10 is an illustration of a side (top) and top (bottom) views of hand movements during a trial of one or more motor assessments.

FIG. 11 is an illustration of hand velocity associated with the hand movements depicted in FIG. 10.

FIG. 12 is a simplified illustration of an exemplary experimental protocol.

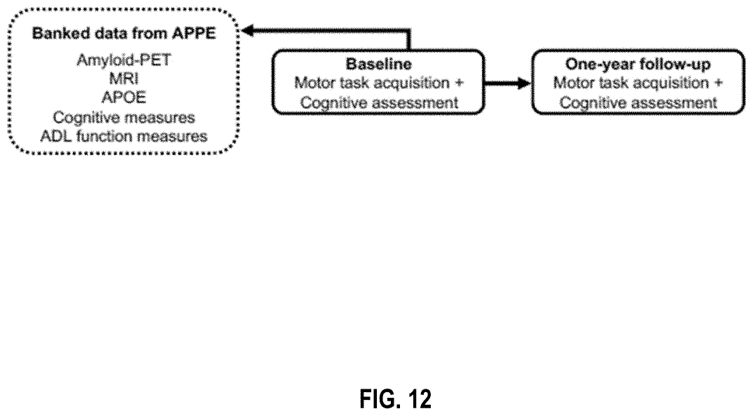

FIG. 13 is a simplified block diagram of an exemplary computing device that may be configured to implement various functions and processes described herein.

Corresponding reference characters indicate corresponding elements among the view of the drawings. The headings used in the figures do not limit the scope of the claims.

### DETAILED DESCRIPTION

The present inventive concept relates to examples of methods, devices, kits, and/or a computer-implemented system for computing a motor test score from test data generated by an individual conducting one or more predetermined motor tasks. In general, a motor task as described herein includes the engagement by the individual with a plurality of test or testing elements according to a predetermined spatial sequence; the details of which may be provided to the individual via an instruction or otherwise. In some examples, the test data includes multiple datasets; each dataset associated with a trial or completion of the motor task. A given dataset may include at least a time and a score associated with the individual's completion of the motor task for each trial according to the spatial sequence.

Examples of the motor task may include a plurality of goal directed movements to visible target locations according to some spatial sequence. The target locations may be spatially arranged such that at least one target is located ipsilateral to the reaching extremity, at least one target is located contralateral to the reaching extremity, and one target is located along the individual's midline. The sequence may define a particular order and manner by which the individual engages target locations and transports the one or more objects for each trial. In addition, the target locations may be spatially positioned and arranged such that the individual can reproduce the sequence consistently for each trial. Variability of the individual's performance in completing the motor tasks according to a predetermined sequence can indicate a neurological change and/or concern.

In many examples, a processor is configured to aggregate the datasets of the test data and compute the motor test score by, e.g., computing a standard deviation of the plurality of test scores over a predetermined time period. The motor test score may reflect a potential concern for a neurological condition based upon a predefined score value threshold (e.g., motor test score <11=no concern). In some examples the system includes a user interface executed by a computing device and configured to provide, via the user interface, a stopwatch function and a scoring function that the individual engages to accommodate aggregation of the plurality of trial times and test scores for access by the processor.

The motor test score described herein accommodates detection, diagnosis/prognosis, tracking, or screening for a neurological condition such as early mild cognitive impairment (MCI) or other cognitive concerns and may be computed efficiently and portably. MCI relates to the stage of early memory loss and/or other cognitive ability of a patient (including issues with visual/spatial perception, judgement, or language) while the patient retains the ability to perform most instrumental activities of daily life. The inventive concept described herein is suitable for detection/prognosis of MCI but also any other neurological condition. The term, “neurological condition” as used herein includes, by non-limiting examples, Alzheimer's disease, behavioral variant frontotemporal dementia, corticobasal degeneration, Huntington's disease, Lewy body dementia, mild cognitive impairment, primary progressive aphasia, progressive supranuclear palsy, vascular dementia, Parkinson's disease, William's syndrome, autism, or a history of stroke. The term further includes traumatic brain injury (TBI), and concussions (mTBI), and other conditions described herein.

Embodiments Associated with Prognosis/Diagnosis of a Neurological Condition:

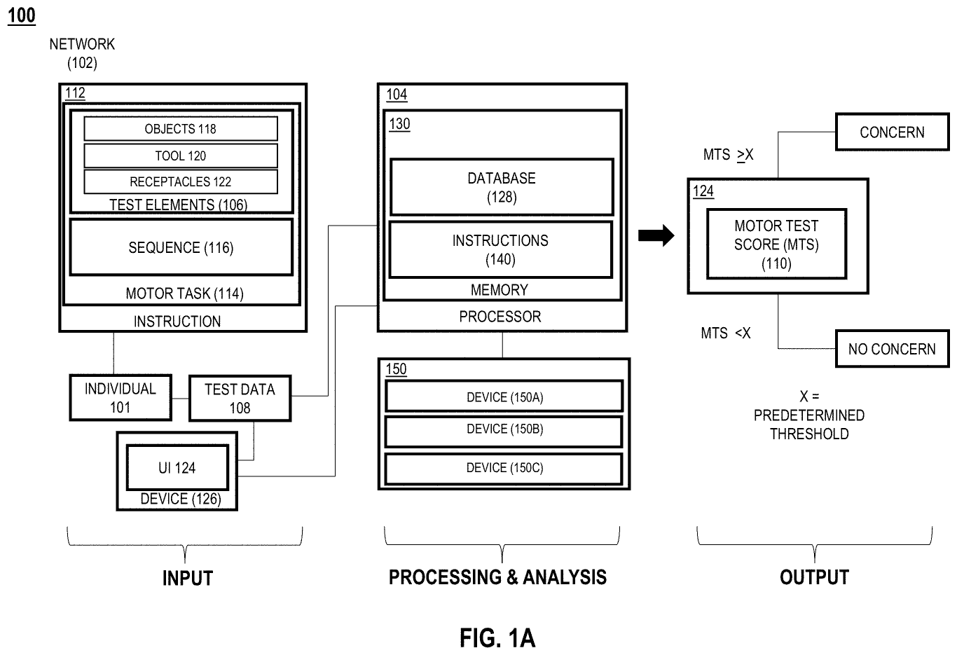

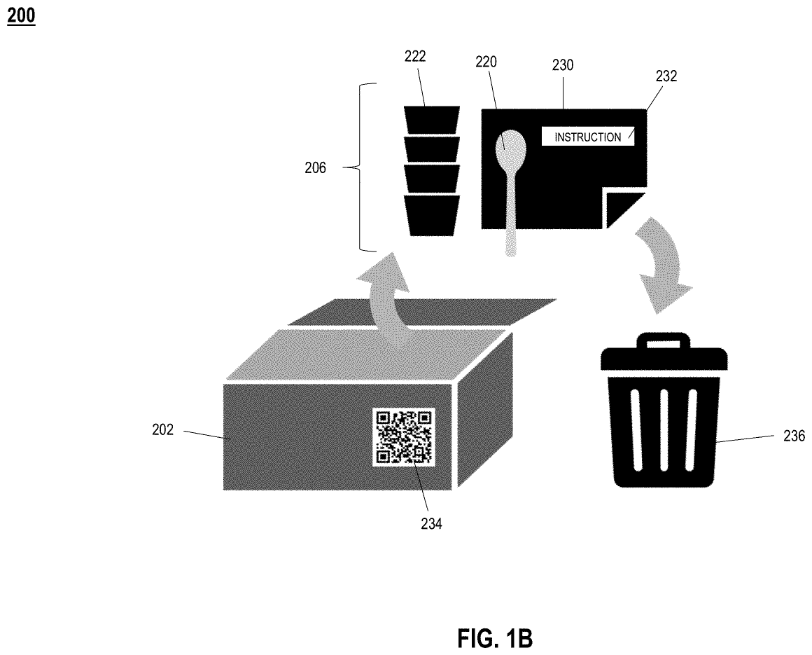

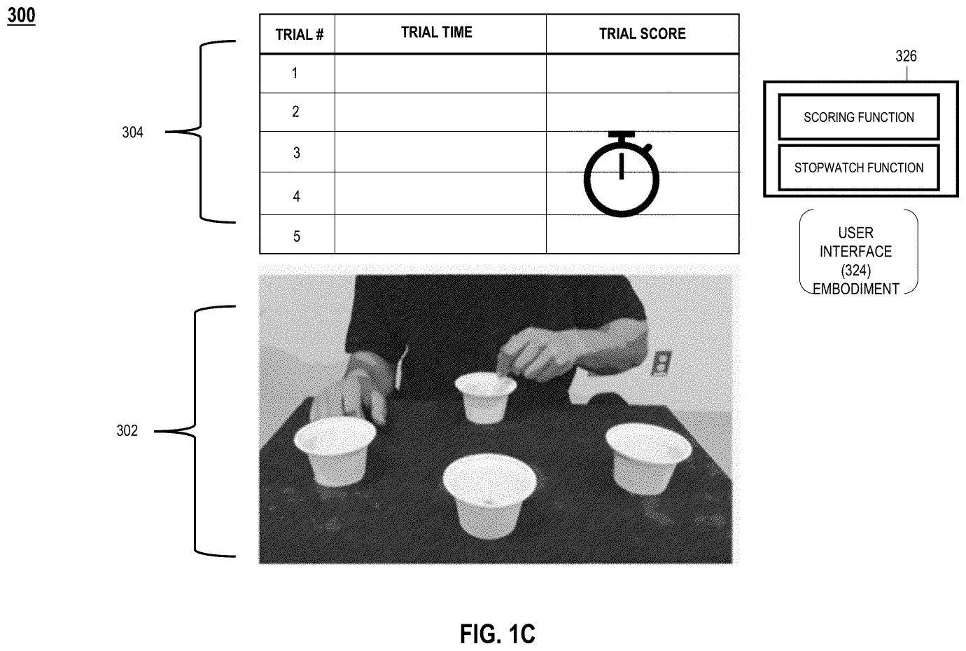

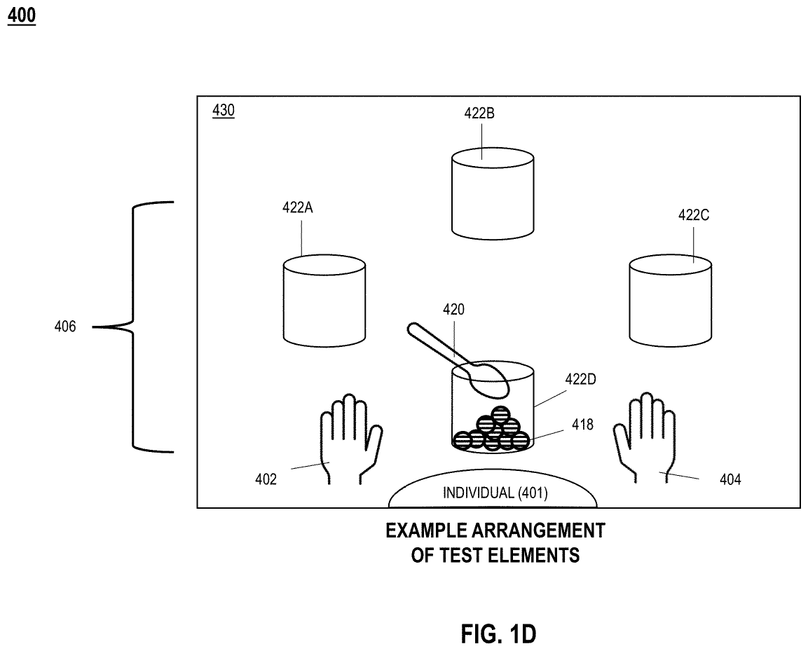

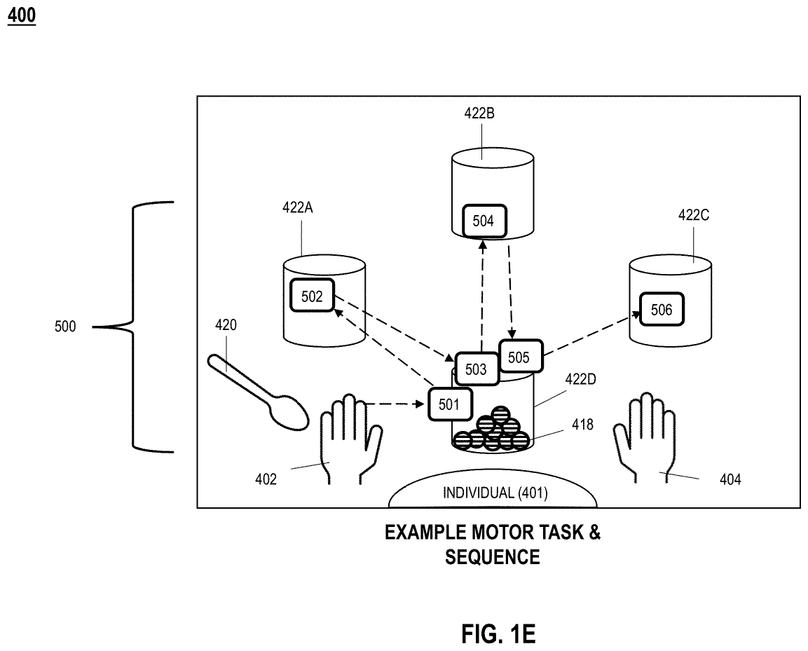

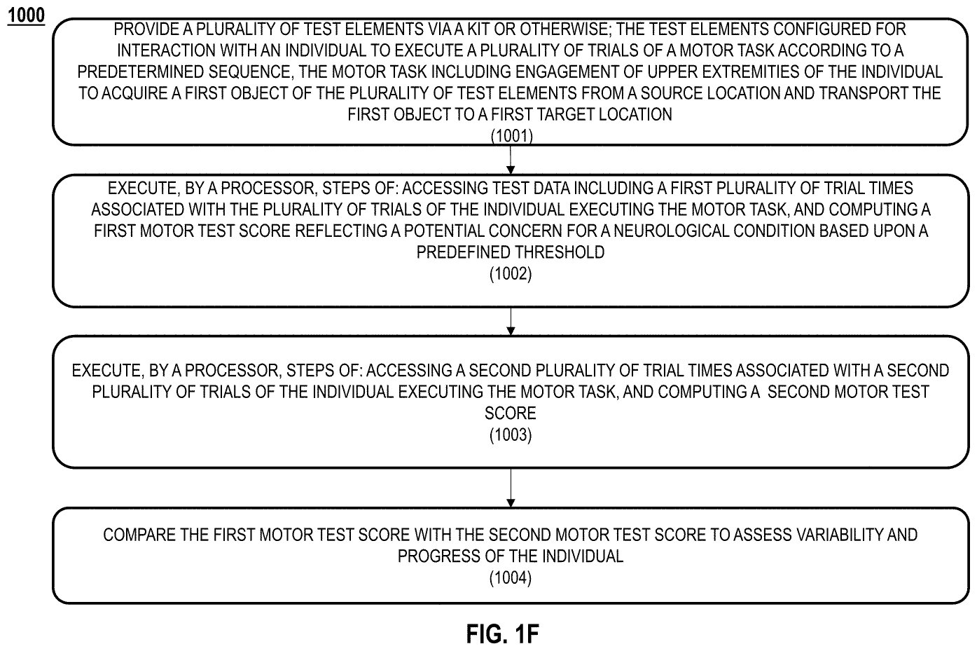

Referring to FIG. 1A, one non-limiting general embodiment of a computer-implemented system 100 is shown for administering one or more motor tasks or motor assessments to one or more of an individual 101. The system 100, defining an input portion, analytics and processing portion, and output portion as shown, is configured to assess, detect, or otherwise screen for a neurological condition and is supported by a network 102 of exemplary devices and components for computer-implemented motor testing and analysis, as further described herein. In general, the system 100 includes a processor 104 and a plurality of test elements 106 that the individual 101 interacts with to generate test data 108. The processor accesses the test data 108 to compute a motor test score 110 for the individual 101 reflecting a potential concern for a neurological condition. More specifically, the individual 101 is prompted, via an instruction 112 or otherwise, to engage with the plurality of test elements 106 and complete trials of a motor task according to a predetermined spatial sequence 116.

Test data 108 may defined as any information suitable for computing the motor test score 110 for the individual 101 by the processor 104. In some examples, the test data 108 includes multiple datasets; each dataset associated with a trial or completion of the motor task 114 by the individual 101 or multiple individuals. A given dataset may include at least a time and a score associated with the individual's completion of the motor task for each trial according to the spatial sequence 116 (FIG. 1C).

The plurality of test elements 106 may include one or more objects 118, a tool 120, and a plurality of receptacles 122. In these embodiments, examples of the motor task 114 may include applying the tool 120 to execute a plurality of goal directed movements of the objects 118 to visible target locations, such as to within or around the receptacles 122. The goal directed movements may be defined by the sequence 116 as further described herein. The target locations and/or the receptacles 122 may be spatially arranged such that at least one target location is located ipsilateral to the reaching extremity, at least one target is located contralateral to the reaching extremity, and one target is located along the individual's midline. The sequence 116 may define a particular order and manner by which the individual 101 engages target locations and transports the one or more objects 118 for each trial. In addition, the receptacles 122 may be spatially positioned and arranged according to predetermined target locations such that the individual 101 can reproduce the sequence 116 consistently for each trial. Variability of the individual's performance in completing the motor task 114 according to the sequence 116 can indicate a neurological change and/or concern.

In many examples, the processor 104 is configured to aggregate datasets of the test data 108 and compute the motor test score 110 by, e.g., computing a standard deviation of the plurality of test scores for the individual 101 over a predetermined time period. The motor test score 110 may reflect a potential concern for a neurological condition based upon a predefined score value threshold (e.g., motor test score <11=no concern, whereas motor test score being equal to or greater than 11 suggests a concern). In some examples the system 100 includes a user interface 124 executed by a computing device 126 and configured to provide, via the user interface, a stopwatch function and a scoring function (FIG. 1C) that the individual 101 engages to accommodate aggregation of the plurality of trial times and test scores of the test data 108 for access by the processor 104.

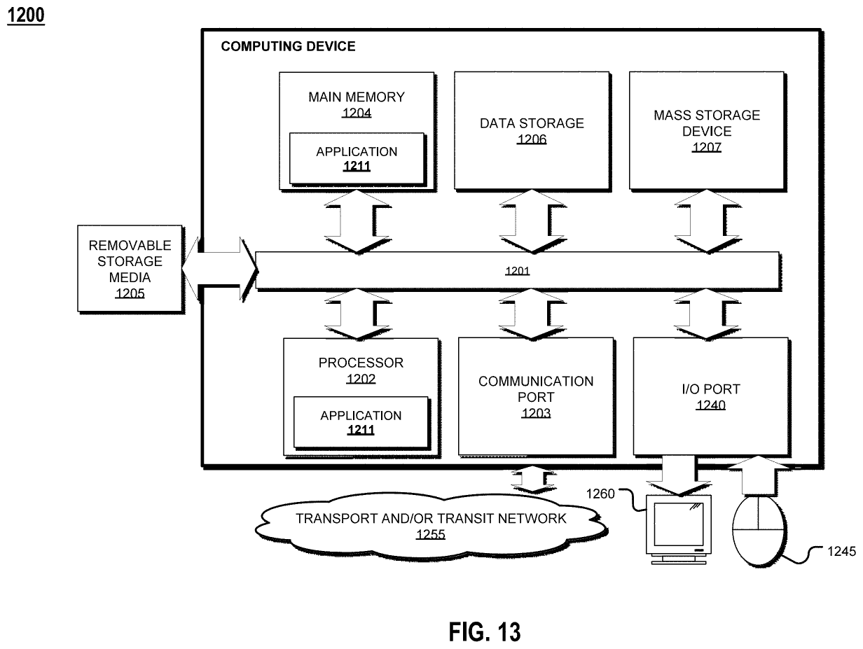

The processor 104 may be implemented via any computing device, cloud computing environment, or other supporting hardware (e.g., computing device 1200 of FIG. 13). The device 126 may include any computing device or similar hardware device capable of generating and or providing test data 108 to processor 104. By non-limiting examples, the device includes any mobile device such as laptops, tablets, smartphones and the like, and may also include any computing device, server, or similar hardware component that can receive/access and transmit data to the processor 104. The processor 104 may further be in operable communication with one or more of a database 128 stored in some memory 130 or storage device. The processor 104 accesses the test data 108 from the device 126 or otherwise, and the test data 108 may be organized and stored in the database 128 for analysis including prognosis prediction functions, as further described herein.

As further shown, the processor 104 accesses and executes instructions 140 that configure the processor 104 to access the test data 108 from the device 126 or other computing devices, execute commands to other devices, and otherwise perform operations for motor testing and analysis as described herein. The processor 104 may be implemented via one or more computing devices and may include any number of suitable processing elements. The instructions 140 may further define or be embodied as code and/or machine-executable instructions executable by the processor 104 that may represent one or more of a procedure, a function, a subprogram, a program, a routine, a subroutine, a module, an object, a software package, a class, or any combination of instructions, data structures, or program statements, and the like. In other words, aspects of the motor testing functionality described herein may be implemented by hardware, software, firmware, middleware, microcode, hardware description languages, or any combination thereof. When implemented in software, firmware, middleware or microcode, the program code or code segments to perform the necessary tasks (e.g., a computer-program product) of the instructions 140 may be stored in a computer-readable or machine-readable medium (e.g., main memory 1204 of FIG. 13), and the processor 104 performs the tasks defined by the code.

Accordingly, the instructions 140 configure the processor 104 to perform operations for motor testing and analysis of the individuals 101, including, e.g., preprocessing the test data 108 and computing the motor test score 110 from the test data 108, as further described herein. The motor test score 110 may be provided or otherwise made accessible to a computing device of the individual 101 or another end user, and may include a numerical value reflecting a cognitive “concern” or “no concern” as indicated in FIG. 1A.

As indicated, the processor 104 may further be in operable communication with a plurality of motor analysis devices 150 (designated by example as device 150A, device 150B, and device 150C). Motor analysis devices 150 include any number of types of devices suitable for supplementing cognitive testing, obtaining or supplementing test data 108, or to further assess motor function of the individual 101 according to various other examples of the system 100 described herein. For example, motor analysis devices 150 may include testing equipment to assess grip strength (via dynamometry) or other motor tests (e.g., gait speed), any number of sensors, and may include computerized testing.

Referring to FIG. 1B, as indicated, aspects of the input portion of the system 100 may be embodied as a kit 200, such that the kit 200 includes a plurality of test elements 206 as indicated, which may be implemented as one example or possible selection of the test elements 106 of the system 100. A kit embodiment of the present concept may be particularly advantageous for deploying the inventive concept as a portable (optionally disposable) package in a primary care provider office or other such location. In general, the kit 200 is suitable for rapid screening, and includes by non-limiting examples, a container 202, such as a box defining a housing or cavity and cover for secure storage and transportation of the plurality of test elements 206, a spoon 220, a plurality of receptacles 222, and optionally one or more moveable objects (not shown), such as beans. The kit 200 may further include a mat 230 with one or more of an instruction 232 imprinted thereon to inform as to a predetermined motor task for engaging the plurality of test elements 106.

Referencing the instruction 232 of the kit 200 or otherwise, an individual (and/or clinician) may be instructed to perform one or more motor tasks and generate test data, such as the test data 108, for each instance of a motor task completed by the individual. The motor task may involve any engagement by the individual with any of the receptacles 222, using the spoon 220 or without the spoon 220, to test motor function by engagement of the upper extremities. In some examples, the instruction 232 may be imprinted upon an exterior side of the container 202, example shown as exterior side instruction 234. As indicated, exterior side instruction 234 may be imprinted in the form of an RFID or bar code, or other such machine-readable image that may provide instructions as to a predetermined motor task for engaging the plurality of test elements 206 via a spatial sequence. In some examples, the kit 200 may be disposable as described and may be entirely discarded within a waste basket 236 after use.

FIG. 1C illustrates a simple example 300 embodiment of the system 100 for generating the test data 108 shown in FIG. 1A, test data designated in FIG. 1C as test data 308. As indicated, test data 304 may be generated for an individual executing trials of a motor task 302. Test data 304 may define one or more datasets for each trial, including a trial time, and a trial score for each execution of the motor task 302 by the individual. Computing device 326 is illustrated to describe further aspects of a user interface (UI) 324 embodiment of generating the test data 204 and providing the same to the processor of FIG. 1A. As indicated by the example 300, a user interface 324, accessible via a computing device 326, may be implemented to record and track the datasets for trials of the motor task 302. More specifically, the user interface 324 may include a scoring function and a stopwatch function to assist an individual with tracking of data points associated with motor task 302 execution. For example, an individual may activate a digital stopwatch function (available via the UI 324) to start a timer upon commencement of the motor task 302 for first trial, and stop the timer of the stopwatch function to record a trial time for the first trial upon completion/execution o of the motor task 302. The UI 324, accessible via a browser, via an app or application of a mobile device, or otherwise, configures the device executing the same to provide such functions and provide access of the recorded test data to the processor 104 for motor test score 110 computation, or otherwise.

Referring to FIGS. 1D-1E, one specific, non-limiting example of a motor task 400 and associated sequence 500 is illustrated. In FIG. 1D, an individual 401 is instructed (via some instruction) to first arrange a plurality of test elements 406 according to the indicated spatial setup. The plurality of test elements 406 includes a plurality of objects 418 such as beans, a tool such as a spoon for picking up and executing goal-directed movements of the one or more objects 418, and a plurality of receptacles 422 (such as cups) that that may receive the one or more objects 418. In the present setup, three distal receptacles 422A-422C of the plurality of test elements 406 are arranged at a radius of 16 cm at −40°, 0°, and 40° relative to a central receptacle 422D. The spatial (visual) setup shown is advantageous because it requires “richer” or “more diverse” biomechanics of the upper extremity, requires both right and left visual fields (visuospatial systems), and allows for planning (or goal directed movement).

In FIG. 1E, the specific predetermined sequence 500 of individual goal-directed movements for executing the motor task 400 is shown. According to a first movement 501 of the sequence 500, a (right-handed) individual 401 uses a nondominant hand 402 (to avoid ceiling effects) and starts by moving one or more of the objects 418 from the receptacle 422D to the receptacle 422A ipsilateral (same side) of the hand (402) used. According to a second movement 502, the individual then returns to the receptacle 422D (center) to acquire two more objects 418 at a time. The individual is further instructed by a third movement 503 of the sequence 500 to transport the objects 418 to the (middle) receptacle 422B, then to the contralateral receptacle 422C according to movements 505-506. The individual may further be instructed to repeat the sequence 500 four more times for a total of 15 out-and-back movements. Task performance may be recorded as trial time (in seconds, via stopwatch); lower values indicative of better performance. Movement errors, such as dropping of the objects 418 mid-reach may be recorded for analysis (as demonstrated by actual application examples described herein). Participants may execute any number of trials of the motor task 400. Task acquisition may be measured as the amount of variability (inter-subject standard deviation) in performance across the trials, such that higher standard deviations indicate less task acquisition.

Sequencing has tight ties to memory (i.e., hippocampus), and is believed to result in improved prediction of cognitive concerns as compared with single, repetitive movements. The more complex the sequence, the more difficult completion of the sequence may be for the individual executing the same. Consistency of sequence execution for a motor task may also be helpful because then individuals can practice and learn across trials. Scores should generally improve across trials for normal/healthy individuals; however, those who cannot learn can show increased variability indicative of cognitive concern.

Referring to FIG. 1F, one example method associated with the inventive concept of FIGS. 1A-1E is illustrated. In blocks 1001-1002 of FIG. 1F, first test data (108 of FIG. 1A) may be generated for analysis such as computation of a first motor test score for an individual 101 (by, e.g., processor 104 of FIG. 1A). The first test data 108, as described herein, includes trial times associated with execution of respective trials of a motor task defining a predetermined sequence of goal-directed movements (e.g., movement of objects from a source location to a target location). The motor task may be administered a plurality of times (e.g., six times); each time the individual repeating the sequence.

In some examples, the motor test score 110 is computed by the processor 104 taking the standard deviation of all trial time scores of the first test data associated with each trial of the motor task, or otherwise automatically computing a motor test score 110. The motor test score 110 may include a predefined threshold (“X” in FIG. 1A) that indicates a “concern” or “no concern” to assist a clinician with deciding whether to conduct further motor assessments or cognitive analysis.

As indicated in blocks 1003-1004 of FIG. 1F, variability of the individual can be assessed over time by comparing first and second motor scores computed by the processor 104 as described.

### Various Other Embodiments and Possible Features of the System 100:

As described, examples of the present inventive concept include a tool 120 for use by a subject in a motor task 114. In various embodiments, the tool 120 comprises a handle and a repository, wherein the repository is configured to receive and hold at least one object (118); wherein the handle comprises at least one sensor configured to collect data and a timer. In embodiments, the at least one sensor comprises a pressure sensor, a skin conductance sensor, or a combination thereof. The pressure sensor can be configured to measure changes in a grip force during the motor task. In embodiments, the skin conductance sensor is configured to measure electrodermal response due to physiological arousal.

In certain embodiments, the at least one sensor is configured to transmit the data to an application running on a processor of a mobile computing device. The application can be configured to compare the data with aggregate patient data.

Another aspect of the inventive concept comprises a system for diagnosis or prognosis of a neurological condition in a subject. In embodiments, the system comprises a tool configured to receive, hold, and manipulate at least one object during a motor task, wherein the tool comprises a timer and at least one tool sensor. The system can further comprise a home receptacle configured to receive and hold the at least one object and a target receptacle configured to receive and hold at least one object. In embodiments, the system the at least one sensor comprises a pressure sensor, a skin conductance sensor, or a combination thereof wherein the pressure sensor is configured to measure changes in a grip force during the motor task and wherein the skin conductance sensor is configured to measure electrodermal response due to physiological arousal. The system can also include a support board configured to support the home receptacle and target receptacle thereon. The support board can comprise an optical hand tracking module configured to record bodily movements during the motor task. In certain embodiments, the system comprises an eye tracker configured to measure pupil dilation throughout the motor task. In certain embodiments, a bottom surface of the home receptacle, the target receptacle, or both comprises an object pressure sensor that is configured to detect the presence or absence of at least one object during the motor task.

In various embodiments, the neurological condition comprises Alzheimer's disease, behavioral variant frontotemporal dementia, corticobasal degeneration, Huntington's disease, Lewy body dementia, mild cognitive impairment, primary progressive aphasia, progressive supranuclear palsy, vascular dementia, Parkinson's disease, William's syndrome, autism, or a history of stroke.

Another aspect of the present inventive concept includes a noninvasive method of predicting hippocampal volume of a subject. In embodiments, the method comprises subjecting the subject to a motor task comprising a plurality of trials, wherein, during each trial, the subject employs a tool as described in any of the various exemplary embodiments disclosed herein to acquire and transport one or more objects at a time from a home receptacle to a plurality of target receptacles and obtaining a task assessment score wherein the task assessment score comprise the variability of time required to complete each trial. In embodiments, a high degree of variability indicates that the subject has a reduced hippocampal volume.

Another aspect of the present inventive concept is a noninvasive method of assessing cortical amyloid deposition a subject. In embodiments, the method comprises subjecting the subject to a motor task comprising a plurality of trials, wherein, during each trial, the subject employs a tool as described in any of the various exemplary embodiments disclosed herein to acquire and transport one or more objects at a time from a home receptacle to a plurality of target receptacles and obtaining a task assessment score wherein the task assessment score comprise the variability of time required to complete each trial. In embodiments, a high degree of variability indicates that the subject has a high degree of cortical amyloid deposition.

Yet another aspect includes a method of pre-screening a subject for a clinical trial. In embodiments, the method comprises subjecting the subject to a motor task comprising a plurality of trials, wherein, during each trial, the subject employs a tool as described in any of the various exemplary embodiments disclosed herein to acquire and transport one or more objects from a home receptacle to a plurality of target receptacles and obtaining a task assessment score wherein the task assessment score comprise the variability of time required to complete each trial. In embodiments, a high degree of variability indicates that the subject should be admitted to the clinical trial.

Another aspect of the present inventive concept includes a method of diagnosing a neurological condition. In embodiments, the method comprises subjecting the subject to a motor task comprising a plurality of trials, wherein, during each trial, the subject employs a tool as described in any of the various exemplary embodiments disclosed herein to acquire and transport one or more objects at a time from a home receptacle to a plurality of target receptacles. The method can further comprise obtaining a task assessment score wherein the task assessment score comprise the variability of time required to complete each trial and diagnosing the subject with the neurological condition if the task assessment score comprises a high degree of variability.

An additional aspect comprises a method of determining a therapeutic efficacy of a drug in treating a neurological condition. In certain embodiments, the method comprises subjecting a subject diagnosed with the neurological condition to a first motor task comprising a plurality of trials, wherein, during each trial, the subject employs a tool as described in any of the various exemplary embodiments disclosed herein to acquire and transport one or more objects at a time from a home receptacle to a plurality of target receptacles. The method further comprises obtaining a first task assessment score wherein the task assessment score comprises the variability of time required to complete each trial of the first motor task. The method can also include administering the drug during a trial treatment period. After the trial treatment period, the method includes subjecting the subject to a second motor task, wherein the second motor task comprises the same steps as the first motor task and obtaining a second task assessment score, wherein the second task assessment score comprises the variability of time required to complete the second motor task. In embodiments, the method further comprises determining that the drug is therapeutically efficacious if the second task assessment score is improved compared to that of the first task assessment score.

Another aspect of the inventive concept includes a method of determining the progression of a neurological condition in a subject. In various embodiments, the method comprises subjecting the subject to a first motor task comprising a plurality of trials, wherein, during each trial, the subject employs a tool as described in any of the various exemplary embodiments disclosed herein to acquire and transport one or more objects at a time from a home receptacle to a plurality of target receptacles. The method can further comprise obtaining a first task assessment score wherein the task assessment score comprises the variability of time required to complete each trial of the first motor task. In embodiments, the method comprises the step of permitting an assessment time period to pass, subjecting the subject to a second motor task, and obtaining a second motor task assessment score following the assessment period, wherein the second motor task comprises the same steps as the first motor task, and determining progression of the neurological condition.

In various embodiments, of the methods described herein, the neurological condition comprises Alzheimer's disease, behavioral variant frontotemporal dementia, corticobasal degeneration, Huntington's disease, Lewy body dementia, mild cognitive impairment, primary progressive aphasia, progressive supranuclear palsy, vascular dementia, Parkinson's disease, William's syndrome, autism, or a history of stroke.

In various embodiments of the methods described herein, the motor task comprises three target receptacles. Each of the three target receptacles can be positioned along a radius surrounding the home target such that each of the three target receptacles are equidistant from the home target. In one embodiment, the first receptacle is placed at −40° along the radius in relation to the home receptacle, the second receptacle is placed at 0° along the radius in relation to the home receptacle, and the third receptacle is placed at 40° along the radius in relation to the home receptacle.

In certain embodiments of the methods disclosed herein, the task assessment score comprises the time required to complete one or more trials of the motor task, changes in motor task performance from across two or more trials, the time required to remove an object and from the home receptacle and deposit the object in the target receptacle, the pressure applied by a subject when holding or manipulating the tool, the number of grip changes during a trial, the number of movement errors during a trial, the number of times an object is dropped during a trial, the angle of the tool during a trial, skin conductance during a trial, the position of the subject's hands, or a combination thereof.

In one embodiment, the one or more target receptacles are oriented radially around the home receptacle. In embodiments, the target receptacles are placed at an even distance from the home receptacle at varying degrees around a single radius. In one non-limiting embodiment, the first receptacle is placed at −40° along the radius in relation to the home receptacle, the second receptacle is placed at 0° along the radius in relation to the home receptacle, the third receptacle is placed at 40° along the radius in relation to the home receptacle, or a combination thereof. The various receptacles can be placed at any location along the radius.

In embodiments, the radius is up to about 50 cm from the center of the home receptacle. The radius can be positioned up to about 30 cm from the center of the home receptacle. The radius can be up to about 20 cm from the center of the home receptacle. In various embodiments, the radius is about 5 cm, about 6, cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, or about 25 cm from the center of the home receptacle.

In embodiments, each of the home and target receptacles are the same size. In alternate embodiments, at least one of the receptacles is a different size. In embodiment, the receptacle comprises a height of up to about 20 cm. The receptacle can comprise a height of up to about 20 cm, up to about 10 cm, up to about 5 cm, up to about 4 cm, up to about 3 cm, up to about 2 cm, or up to about 1 cm. The height of at least one receptacle can be about 4.5 cm, about 4.6 cm, about 4.7 cm, about 4.8 cm, about 4.9 cm, about 5.0 cm, about 5.1 cm, about 5.2 cm, about 5.3 cm, about 5.4 cm, about 5.5 cm, about 5.6 cm, about 5.7 cm, about 5.8 cm, about 5.9 cm, about 6.0 cm, about 6.1 cm, about 6.2 cm, about 6.3 cm, about 6.4 cm, or about 6.5 cm. In one embodiment, the height of at least one receptacle is about 5.8 cm.

In embodiments, the receptacle can comprise a diameter of up to about 50 cm. In embodiments, the receptacle comprises a diameter of up to about 40 cm, up to about 30 cm, up to about 20 cm, up to about 10 cm, or up to about 5 cm. The diameter of the receptacle can be as small as 1 cm. The diameter of the at least one receptacle can be about 8.5 cm, about 8.6 cm, about 8.7 cm, about 8.8 cm, about 8.9 cm, about 9.0 cm, about 9.1 cm, about 9.2 cm, about 9.3 cm, about 9.4 cm, about 9.5 cm, about 9.6 cm, about 9.7 cm, about 9.8 cm, about 9.9 cm, or about 10 cm. In one embodiment, the diameter of at least one receptacle can be about 9.5 cm.

In one embodiment, the subject moves the object sequentially from one receptacle to the next receptacle. The motor task can require that the subject manipulate the tool such that the object is moved serially from a first receptacle to a second receptacle and from the second receptacle to a third receptacle. In another embodiment, the motor task requires that the subject manipulate the tool such that the one or more objects are moved serially from a first receptacle to a second receptacle, from the second receptacle to a third receptacle, and from a third receptacle to a fourth receptacle. In certain embodiments, the motor task requires that the subject manipulate the tool such that the one or more objects are moved from one receptacle in a non-serial manner (e.g. from the second receptacle to the first receptacle and then from the first receptacle to the third receptacle). In embodiments, the first move can be into any one of the various receptacles and the second move can be made to any one of the remaining receptacles. This process can be repeated until each of the receptacles have received at least one object. In embodiments, the subject is instructed to move from one receptacle to the next as quickly as possible.

In one embodiment, the subject uses the tool to acquire and move one or more objects from one receptacle to another. The subject can be required to use the tool to acquire and move more than one object at a time from one receptacle to another. In an embodiment, the subject uses the tool to acquire and move two objects at a time from one receptacle to another. The subject can move up to ten objects at a time. In embodiments, the subject moves one, two, three, four, five, six, seven, eight, nine, or ten objects at a time.

The time taken to complete the motor task can be assessed. In embodiments, the time required to complete each trial can be assessed. In embodiments, the motor task comprises up to 150 trials. The motor task can comprise up to 100, 90. 80, 70, 40, 50, 40 30, 20, or 10 trials. In embodiments, the motor task comprises as few as a single trial. The motor task can comprise up to ten trials. In certain embodiments, the motor task comprises one, two, three, four, five, six, seven, eight, nine, ten, eleven, twelve, thirteen, fourteen, fifteen, sixteen, seventeen, eighteen, nineteen, or twenty trials.

Embodiments can comprise up to twenty receptacles. Certain embodiments comprise a single receptacle. Embodiments can comprise up to ten receptacles. The motor task can employ one, two, three, four, five, six, seven, eight, nine, or ten receptacles. One embodiment employs four receptacles.

In certain embodiments, the object comprises a rounded object. The object can comprise a ball, a marble, a bean, a pea, or any combination thereof. In embodiments, the object comprise a volume of up to about 5 cm3. Each object can comprise a volume as small as about 0.1 cm3. In embodiments, the object comprises a volume of about 0.1 cm3, about 0.2 cm3, about 0.3 cm3, about 0.4 cm3, about 0.5 cm3, about 0.6 cm3, about 0.7 cm3, about 0.8 cm3, about 0.9 cm3, about 1 cm3, about 2 cm3, about 3 cm3, about 4 cm3, or about 5 cm3.

In embodiments, the tool can comprise any apparatus that is capable of retrieving objects form a cup or receptacle. In one embodiment, the tool comprises a spoon.

In an embodiment, the motor task comprises one more receptacles, one or more objects, one or more tools, written instructions, a support board or combination thereof. In embodiments, the support board comprises markings to provide a visual guide for placement of the receptacles. The motor task can further comprise an adhesive to reversibly adhere the support board to a table. FIG. 1C provides an example of the motor task under one embodiment.

In operation, the subject can begin the motor task by using the tool to remove one or more objects from the home receptacle and manipulate the tool to move the one or more objects from the home receptacle to the first target receptacle. In embodiments with more than one target receptacle, the subject returns with the tool to the home receptacle to retrieve and remove a second set one or more objects and manipulates the tool to move the second set of one or more objects to the second target receptacle. This process can be repeated until the subject successfully deposits one or more objects into each of the target receptacles. In embodiments, a single trial is completed when the subject successfully deposits one or more objects into each of the target receptacles and repeats the process a predetermined number of times. A trial can comprise deposition of one or more objects into each of the targets only once. In alternate embodiments, a trial requires multiple rounds of depositing one or more objects into each of the receptacles. A trial can comprise up to 50 rounds, up to 40 rounds, up to 30 rounds, up to 20 rounds, up to 10 rounds, up to 15 rounds, up to 5 rounds, up to 4 rounds, up to 3 rounds, up to 2, rounds or a single round. A trial can be completed when all of the objects have been removed from the home receptacle. The motor task can be completed when the subject has completed the required number of trials.

At the beginning of the motor task, the home receptacle can comprise one or more objects. At the beginning of the motor task, the home receptacle can comprise up to about 100 objects. The home receptacle can comprise up to about 50 objects at the beginning of the motor task. In embodiments, the home receptacle comprises up to about 45 objects, up to about 40 objects, up to about 35 objects, up to about 30 objects, up to about 25 objects, up to about 20 objects, up to about 15 objects, up to about 10 objects, or up to about 5 objects.

In embodiments, different trials can be performed using the non-dominant or dominant hands. In one embodiment, four trials are completed with the non-dominant hand (to evaluate a practice effect), and one trial is performed with the dominant hand.

As used herein, a “task assessment score” can refer to data obtained during the motor task and interpretations of the same. In embodiments, the task assessment score comprises the time required to complete one or more trials of the motor task. In embodiments, the task assessment score comprises the variability in the time required to complete the motor task over multiple trials (also referred to herein as “task acquisition). The task assessment score can comprise changes in motor task performance across two or more trials. The task assessment score can comprise the time required to remove an object and from the home receptacle and deposit the object in the appropriate target receptacle. Task assessment scores can comprise the pressure applied by a subject when holding or manipulating the tool, the number of grip changes during a trial, the number of movement errors during a trial, the number of times an object is dropped during a trial, the angle of the tool during a trial, skin conductance during a trial. The task assessment score can comprise the position of the subject's joints, hands, wrists, elbows, or a combination thereof during a trial.

In certain embodiments the task assessment score comprises the time required to complete a performance trial following one or more practice trials. The task assessment score can comprise the time required to complete a performance trial following one, two, three, four, five, six, seven, eight, nine, or ten practice trials. In certain embodiments, the performance trial comprises the second, third, fourth, fifth, sixth, seventh, eight, ninth, tenth, or eleventh trial. In one embodiment, the task assessment score comprises the time required to complete a performance trial following three practice trials (i.e., the performance trial is the fourth trial). In one embodiment, a subject's performance trial time of about 60 seconds or more following three practice trials indicates that the subject is about 5 times more likely to suffer from cognitive impairment than a subject with a task assessment score of less than about 60 seconds. In one embodiment, a subject's trial time of 68 seconds or more on the fourth trial indicates that the subject is about 4.76 times more likely to be amyloid positive than a person with a task assessment score of below 68 seconds.

In general, the system accommodates more efficient screening of cognitive impairment or other neurological conditions, and the system can be, e.g., deployed swiftly prior to examination by a primary care physician or their medical staff to determine when to refer patients for further neuropsychological examination for early cognitive decline or other neurological concerns. In some embodiments, the system includes an interactive digital interface (e.g., application, and/or interactive website) that includes the following pages and/or functions:
1) a landing page that describes the motor assessment
2) FAQs about the assessment
3) an embedded stopwatch to time each trial of the assessment
4) a function that automatically scores the assessment after 6 (or fewer) trials. The scoring will be based on threshold scores set by the inventor

In some examples, the system 100 and associated methods combines the digital stopwatch and scoring function into a single digital platform (e.g., website, application, etc.) along with information about the assessment to streamline and improve upon existing cognitive and motor testing technologies. The system may be offered as a kit including testing components and providing access to devices for engaging the digital user interface while the patient interacts with the testing components. The system may be offered in multiple languages, may track progress of individual patients, and other features which are fully appreciated and consistent with the spirit and scope of the inventive concept.

### Other Embodiments

While the inventive concept has been described in conjunction with the detailed description thereof, the foregoing description is intended to illustrate and not limit the scope of the inventive concept, which is defined by the scope of the appended claims. Other aspects, advantages, and modifications are within the scope of the following claims. The inventive concept will be further described in the following further examples, which do not limit the scope of the inventive concept described in the claims.

### FURTHER EXAMPLES

Examples are provided below to facilitate a more complete understanding of the present inventive concept. The following examples illustrate exemplary, non-limiting possible applications and/or aspects of the inventive concept such as the system 100, kit 200, and method 1000. However, the scope of the inventive concept is not limited to specific embodiments disclosed in these Examples, which are for purposes of illustration only, and alternative methods can be utilized to obtain similar results.

### Example 1

Motor behavior (i.e., human movement) may be used to predict dementia progression and Alzheimer's disease biomarkers in home.

There is a newfound interest in using motor assessments to evaluate cognitive abilities, particularly in older adults. However, most established motor assessments require extensive lab-based instrumentation or technology, and do not necessarily correlate with cognitive function or disease biomarkers. Moreover, most assessments require a clinician or researcher to administer them. We therefore developed this system using inexpensive items that could be mailed to a person to perform in their home.

In one embodiment, this system involved participants using a plastic spoon to scoop two beans (kidney, raw) at a time from a center proximal “start” cup and transport them as quickly as possible to three distal “target” cups as fast as possible. These cups can be single-serving plastic yogurt cups. The cups can be secured to a printed paper support board or mat. In one embodiment, the cups are secured using double-sided adhesive. The cups can be oriented at 45°, 90°, and 135° around the start cup at a distance of 16 cm. The start cup is oriented along the participant's midline in front of the seated participant. This means that this motor task has no balance requirements, such that even balance-impaired individuals can complete it by themselves with minimal risk. Participants are instructed to move as quickly yet as accurately as possible, first to the left target cup, next to the center target cup, and then to right cup. They repeat this sequence five times to complete the trial. Participants can time themselves and record their performance on a scoresheet provided. Each trial begins when the participants pick up the spoon and ends when they drop the last two beans into the cup. Participants will self-report any errors in performance as well. One measure of performance can be the time taken to complete the task (i.e., “trial time”), with faster times indicating better performance. In embodiments, a measure of performance includes the number or frequency of errors during the motor task, wherein a reduced number of errors are associated with better performance. A measure of performance can include monitoring the path taken between cups, wherein a straighter or more direct path is indicative of good performance. In certain embodiments, the measure of performance includes the time taken to complete the task, the number of errors, the directness of the path taken between cups, or a combination thereof, wherein a fast trial time with few errors and a direct path indicates very good performance, a slow trial time with multiple errors and an indirect path indicates poor performance. The element of the research is the compiling of the yogurt cups, plastic spoon, beans, support board, and scoresheet into a single, mailable kit. An online portal can also be employed wherein subjects or patients report their times for data collection.

This system uses functional yet fun human movement to gain insight into Alzheimer's disease and dementia. This was designed based on the need for simpler, more affordable methods for evaluating human movement that could be done in peoples' homes.

Exemplary design features include: an instrumented spoon that allows players to time themselves while tracking their movement throughout the process. Other exemplary embodiments include the following:

Technology-Based Advances
1. Pressure (piezoelectric) sensor in the spoon handle to measure changes in grip force during trial
2. Pressure sensor embedded on the bottom of each cup to estimate when beans are removed in placed within each cup during a trial. Sends timestamp to stopwatch to register when changes in pressure within each cup occur. Exemplary sensors comprise a force sensing resistor. One non-limiting exemplary pressure sensor comprises the FSR02CE sensor available from HEICO OHMITE, L.L.C., Warrenville, Ill., USA.
3. Gyroscope embedded in the spoon handle to record angle of the spoon during trial.
4. Optical hand tracking module embedded in support board to record position and joint angles of the hand, wrist and elbow. One non-limiting, exemplary hand tracking module is the Leap Motion Controller (available from UltraLeap, Bristol, United Kingdom).
5. Skin conductance sensor placed in handle of sensor to measure electrodermal response due to physiological arousal.
6. Eye tracker to measure pupil dilation throughout the trial to gauge cognitive load at different times of the trial, One non-limiting, exemplary example of a suitable eye tracker is a pair of Tobi Pro Glasses (available from Tobii AB AKTIEBOLAG, SWEDEN).

Structural Advances
1. Improved durability and/or sustainability of materials
   A. Thicker plastic cups
   B. Cups with more recycled material
   C. Different shaped spoons, such as a standard spoon shape, a tablespoon shape, a teaspoon shape, a soup spoon shape, a flat and shallow spoon, or a deep and round spoon
   D. Heavier spoons
   E. Recyclable/compostable spoons (they are not going in any liquid or food so they do not need to be durable plastic, per se)
2. Improved durability/appearance of ‘beans’
   F. More uniform in shape
   G. Glossier finish
   H. Different colors
3. Stopwatch embedded in spoon (so player could squeeze the handle to start and stop the trial)

Preliminary testing indicates that our system and methods can be done in-home and unsupervised, and better predicts disease progression than existing cognitive and functional measures, which are administered by a clinician. Our preliminary testing also suggests that our disclosure is more culturally sensitive and less biased against minorities than existing dementia assessments.

Could be purchased by pharmaceutical companies to use as an enrichment strategy for recruiting participants into drug clinical trials; Could be sold to clinicians to better predict dementia progression; Could be sold to individual users to better track brain health. There are believed to be no available alternatives or competition.

### Example 2

Remote, Unsupervised Functional Motor Task Evaluation in Older Adults Across the United States Using the MindCrowd Electronic Cohort.

Abstract

The COVID-19 pandemic has impacted the ability to evaluate motor function in older adults, as motor assessments typically require face-to-face interaction. This study tested whether motor function can be assessed at home. One hundred seventy-seven older adults nationwide (recruited through the MindCrowd electronic cohort) completed a brief functional upper-extremity assessment at home and unsupervised. Performance data were compared to data from an independent sample of community-dwelling older adults (N=250) assessed by an experimenter in-lab. The effect of age on performance was similar between the in-lab and at-home groups for both the dominant and non-dominant hand. Practice effects were also similar between the groups. Assessing upper-extremity motor function remotely is feasible and reliable in community-dwelling older adults. This test offers a practical solution in response to the COVID-19 pandemic and telehealth practice and other research involving remote or geographically isolated individuals.

Introduction

Assessing motor function in older adults is essential, as it is affected by neurologic conditions like stroke, Mild Cognitive Impairment (MCI) (Jekel et al., 2015), Parkinson's disease (Roalf et al., 2018) and Alzheimer's disease (Kluger et al., 1997). However, most clinical motor assessments require face-to-face administration and specialized medical equipment (e.g., dynamometry) (Milne & Maule, 1984), and experimental measures typically use motion capture (Heath et al., 1999; Owings & Grabiner, 2004), robotics (Pearce et al., 2012), or other expensive technologies (e.g., transcranial magnetic stimulation, electromyography, or magnetic resonance imaging) (Ferber et al., 2002; Resnick, 2000; Schambra et al., 2015). Thus, these motor assessments are not feasible in remote contexts and are limited in re-test frequency or longitudinal evaluation due to cost and time constraints. Due to the COVID-19 pandemic, many medical practices and research methods have shifted to remote, internet-based approaches (Klil-Drori et al., 2021; Rowley et al., 2019; Thornton, 2020), and many older adults are unwilling or unable to engage in face-to-face, in-person research (Roe et al., 2021). However, the objective motor assessments currently available cannot be done remotely due to instrumentation or supervision requirements (i.e., a piece of equipment and a test administrator is needed). These limitations are problematic for evaluating motor function, tracking disease progression over time, and measuring the efficacy of an intervention as an outcome variable, particularly for older populations who have been encouraged to remain isolated due to COVID-19. Thus, there is an urgent need for objective motor assessments that are feasible and reliable for remote administration in people's homes.

A simple, low-cost upper extremity motor task was developed as a more accessible and affordable. It is fabricated from household items, requires minimal technology (only a stopwatch or timing device), and can be assembled and mailed for <$10. Research on the face-to-face version of the task (administered by an experimenter) has shown that older adult task performance is associated with cognitive status, visuospatial memory and a one-year decline in activities of daily living, yet is not subject to sex differences. It is also feasible for stroke, Parkinson's disease, and MCI populations. These features collectively make this task amenable to a remote, at-home setting, but it has not been validated for such use to date.

To test the reliability of our motor assessment as an unsupervised, at-home tool, we utilized the MindCrowd electronic cohort (Huentelman et al., 2020). MindCrowd is an internet-based platform focused on neuroaging research that has collected participant data via online surveys and a brief cognitive assessment, as well as remote genotyping via mail (Talboom et al., 2019). Thus, the purpose of this study was to leverage the MindCrowd infrastructure to validate a remote, unsupervised version of our motor assessment within an in-home setting. To do so, we utilized a subsample of MindCrowd participants over age 40, and compared their at-home, unsupervised performance to data from an existing in-lab, supervised sample

Methods

Participants: All participants in this study had no previous history of mental illness, neurologic disease, or injury (i.e., stroke, history of seizures, concussion diagnosis, brain disease, or arthritis of the hands or upper limbs). All participants reported normal visual acuity and absence of any peripheral sensory or motor loss/pathology. Although MindCrowd itself has users worldwide, all participants in this study resided in the United States.

Six hundred seventy-nine participants were recruited to this study via e-mail, which provided an overview of the motor task and the option to agree to participate if interested. If the participant consented (via e-mail), a kit containing the motor task, along with instructions for administration and reporting data, was sent to the mailing address provided by the participant. As of January 2021, 241 kits had been mailed to consented participants, and 177 participants (mean age=59.13 years+/−9.18; 132 female) had completed the task and reported their data back to MindCrowd. Thus, ˜⅓ of contacted MindCrowd users were willing to participate, ˜75% of whom completed the assessment with their dominant and non-dominant hand once consented. In this cohort, hand dominance was self-reported. The WCG IRB Institutional Review Board approved this portion of the study.

The MindCrowd data were then compared to an independent sample that had been previously completed collected in-lab (Hooyman et al., 2020) (N=250, mean age=73.12 years+/−8.22, female=129). All participants in the in-lab cohort were assessed on their dominant and non-dominant hand and provided written informed consent before participation following the World Medical Association Declaration of Helsinki. The Arizona State University and Utah State University Institutional Review Boards approved this portion of the study. A subset of 106 participants from the in-lab cohort (mean age=71.29+/−8.67, female=72) also completed three more trials of the motor task with the non-dominant hand to evaluate a practice effect. Hand dominance in this cohort was assessed with the Edinburgh Handedness Inventory (Oldfield, 1971).

Motor task: Briefly, task performance involved 15 repetitions of acquiring and transporting two kidney beans (˜0.5 cm3) at a time with a standard plastic spoon from a plastic ‘home’ cup (9.5 cm in diameter and 5.8 cm in height) to one of three ‘target’ cups that were the same size as the home container. The target containers were secured radially around the home container at 40°, 0°, and 40° at 16 cm. At the start of the trial, thirty beans were placed into the home cup (15 repetitions×2 beans/rep). To replicate the experimental set-up at home, individual kits were mailed that had 4 cups, 30 beans, a spoon, written instructions, and a paper ‘support board’ that provided a visual of where the home and target containers should be placed relative to their body, along with tape adhesive to adhere the mat or support board to a table while seated. FIGS. 1C-1D illustrate an assembled view of the motor task. Participants started by reaching to the left target cup, then returned to the central cup to acquire two more beans to transport to the middle target cup, then the right target cup, and then repeated this 3-cup sequence five times for a total of 15 reaches. The trial ended once the last two beans were deposited into the last cup. Performance was measured as the amount of time it took to complete all 15 reaches, i.e., ‘trial time.’ Four trials were completed with the non-dominant hand (to evaluate a practice effect), and one trial was performed with the dominant hand. MindCrowd participants either timed themselves (61%) or were timed by a partner (39%), while an experimenter timed the in-lab participants.

Statistical analyses: All analyses were performed in R (v4.0.0). To determine the reliability of the assessment, we performed a general linear model with task performance (i.e., trial time) as the dependent variable; group (MindCrowd vs. in-lab), sex, age, and hand were included as independent variables. Since the non-dominant hand performed four trials overall, only the first trial was used in this first analysis. To determine the similarity in practice effects of the non-dominant hand between MindCrowd and in-lab groups, we performed an autoregressive linear mixed-effects model with performance of the nondominant hand across the four trials as the dependent variable, and trial number (1-4), group, age, and sex as fixed effects and random intercepts on the participant.

Results

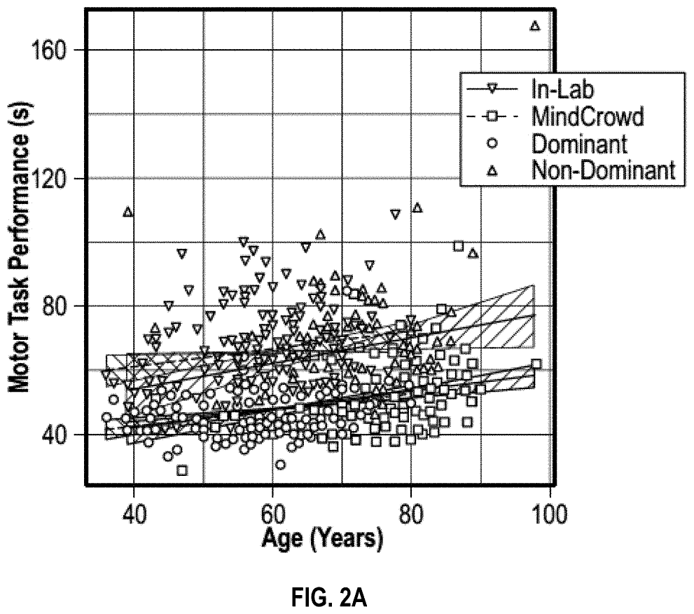

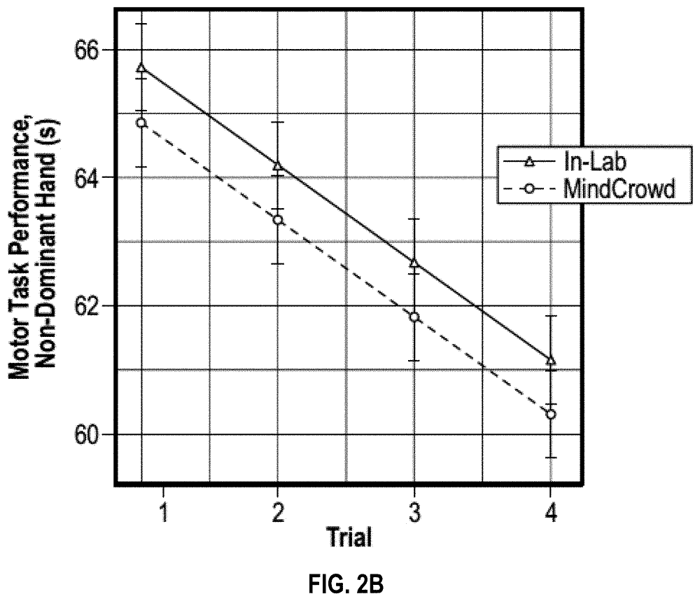

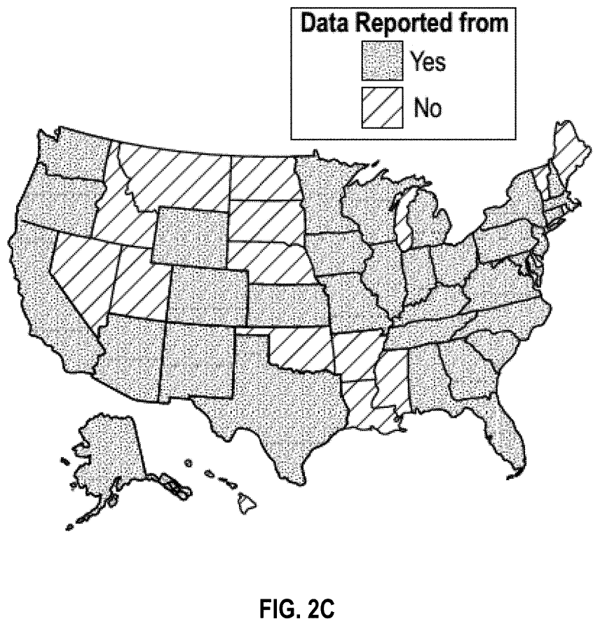

The general linear model showed no significant effect of group (MindCrowd vs. in-lab) on task performance (p=0.2), indicating that data collected in-lab by an experimenter was comparable to data collected at home by the participant unsupervised. For example, the mean difference in task performance between in-lab and MindCrowd groups was only ˜2% (1.3 seconds). Regardless of group, there was a positive relationship between age and task performance (β_{age}=0.29, p<0.0001, 95% CI=[0.2, 0.38]), consistent with (Spedden et al., 2017). There was also an effect of hand dominance on task performance (β_{dominantHand}=−8.62, p<0.0001, 95% CI=[−9.46, −7.78]) (FIG. 2A), consistent with previous data showing that the dominant hand is faster (Schaefer, 2015). Furthermore, there was also no effect of sex on task performance (βSex=1.21, p=0.17, 95% CI=[−0.51, 2.94]), again consistent with previous data (Hooyman et al., 2020). Within the MindCrowd cohort, there was no significant effect on task performance based on whether participants timed themselves or were timed by someone else (mean non-dominant hand difference=2.6 seconds, 95% CI=[−1.7, 6.4], p=0.18; mean dominant hand difference=0.51 seconds, 95% CI=[−1.6, 2.7], p=0.65). These results collectively show that age (and hand used) impact task performance, while the location and level of task supervision do not. The autoregressive linear mixed-effects model also exhibited no effect of group on practice effects when the task is completed multiple times with the non-dominant hand (p=0.12) (FIG. 2B). Consistent with earlier data, there was a significant effect of the trial (i.e., participants improved with practice) (βtrial=−1.8, p<0.0001, 95% CI=[−2.27, −1.32]), but these results indicate that improvements in the motor task due to repeated exposure were also not dependent on location or level of supervision.

Discussion

The purpose of this study was to validate a remote, unsupervised version of a functional motor assessment within an in-home setting. Results showed that motor performance collected in-home without supervision was not significantly different from data collected face-to-face in a laboratory setting. In other words, performance, and corresponding practice effects, measured in the home were not statistically different from those measured in the lab. This suggests that older adults nationwide can reliably perform this motor assessment remotely without supervision or clinical oversight. The feasibility and reliability reported here demonstrate measurable benefits for preclinical research in older adults. First, once participants consented to participate and the kits were mailed, it took only 174 days for all 177 participants to perform the task and report their data. This rate is >1 participant a day, including weekends, whereas the rate of recruitment/participation in a face-to-face research study is often much slower, even before the COVID-19 pandemic. Second, the low cost and simplicity of the individual task components (e.g., beans, plastic spoon) allowed motor data to be collected from all over the United States. As shown in FIG. 2C, data were collected from participants in 33 different states, a paradigm much different than what is feasible in other single-site studies that involve face-to-face assessments. The ability to collect across a geographically distributed sample can make research more inclusive, particularly for older adults who cannot drive or who do not have access to reliable public transportation (Park et al., 2010). Third, social isolation (due to the COVID-19 pandemic or otherwise) can substantially affect depression and psychological distress among older adults (Gorenko et al., 2021); thus, gerontological research must continue pursuing ways to engage and assess isolated older adults. Lastly, the feasibility and reliability of assessing motor function at home and unsupervised allows for clinical trial enrichment. Performance on the motor task used here has been linked to visuospatial processes (Lingo VanGilder et al., 2018, 2019), which have been shown to decline earlier than memory scores in cases of eventual Mild Cognitive Impairment (MCI) diagnosis (Schaefer & Duff, 2017). Furthermore, performance on this motor task improves the prediction of eventual functional decline in confirmed MCI. The remote version of this task would allow researchers or clinicians to monitor or screen individuals easily, regardless of geographical location, for study enrollment or neuropsychological follow-up.

A benefit of electronic cohorts is that they allow for more extensive data to be collected from more distributed samples. MindCrowd has collected a number of demographic, health, and lifestyle variables on its users (see Talboom et al., 2019), although very few of these have been included in the analyses presented here because of limitations in the in-lab sample. In fact, all shared data elements (e.g., age, sex) between the two cohorts were included in the analyses to consider as many covariates as possible. While this is a limitation of this study, this highlights the advantage of leveraging electronic cohorts for human subjects research. They allow for more extensive and distributed samples and enable a much more robust set of data to be collected than typical face-to-face laboratory research. With the remote version of this motor assessment validated, future studies can investigate how other factors, such as zip code, race/ethnicity, socioeconomic status, former occupation, marital status, comorbidities, polypharmacology, and genetics affect motor function and motor decline in older adults.

To summarize, this study showed that this motor assessment could feasibly and reliably be collected remotely without supervision in older adults. We demonstrate that individuals across a range of geographical locations and ages were willing to participate in a study that involved the assembly, completion, and reporting of a task.

### Example 3

Association Between Motor Task Acquisition and Hippocampal Atrophy Across Cognitively Unimpaired, Amnestic Mild Cognitive Impairment, and Alzheimer's Disease Individuals

Abstract

Hippocampal atrophy is a widely used biomarker for Alzheimer's disease (AD), but the cost, time, and contraindications associated with magnetic resonance imaging (MRI) limit its use. Recent work has shown that a low-cost upper extremity motor task can identify AD risk. Fifty-four older adults (15 cognitively unimpaired, 24 amnestic Mild Cognitive Impairment, and 15 AD) completed six motor task trials and a structural MRI. Motor task acquisition significantly predicted bilateral hippocampal volume, controlling for age, sex, education, and memory. Thus, this motor task may be an affordable, non-invasive screen for AD risk and progression.

Introduction

Hippocampal atrophy is a widely used biomarker for Alzheimer's disease (AD) stage and progression. It is measured using magnetic resonance imaging (MRI), which is cheaper, more widely available, or less invasive than other biomarker testing, such as positron emission tomography and lumbar puncture. However, it still requires extensive equipment, staff, and time, and has a number of contraindications that limit its use among older adults specifically.

Factors like claustrophobia or high body mass further restrict MRI use for geriatrics. Older adults are also more likely to move more while in the scanner, affecting scan quality. Furthermore, the need for medical personnel and settings (i.e., hospital) disproportionately discourages under-represented minorities from participating in clinical trials and biomedical research and seeking diagnoses or treatment. Thus, a low-cost, non-invasive, and widely-accessible method to identify hippocampal atrophy in older adults is needed.

Motor behavior can provide a biomarker that addresses these needs, as complex upper-limb movements have been associated with AD severity. Recent work has demonstrated that a rapid, easy-to-administer upper-limb motor task involving adaptive fine motor skill can predict disease progression and is more sensitive to cognitive status than other simple motor assessments while requiring no computer hardware/software. It is feasible for amnestic Mild Cognitive Impairment (aMCI) cohorts to perform, and with repeated exposure it can show within-session practice effects (i.e., motor task acquisition) that indicate intact learning ability (consistent with). This is in contrast to other motor tasks that require technology (e.g., movement sensors, motion capture, electromyography, or transcranial magnetic stimulation) and often show a ceiling effect. Given the task's association with disease progression and cognitive status, this short report tested its relationship with hippocampal volume across the AD spectrum (i.e., cognitively unimpaired, aMCI, and mild AD). Without wishing to be bound by theory, motor task acquisition is related to hippocampal volume, even after controlling for age, sex, education, and memory function.

### Methods

Participants

Fifty-four older adults participated in this study, who were a subset of ClinicalTrials.gov NCT03466736 recruited through April 2019. Fifteen were cognitively unimpaired (CU) (mean±SD age=71.9±4.8 years; 13 females; 17.1±1.8 years of education), 24 were classified with amnestic MCI (aMCI) (mean±SD age=74.1±5.7 years; 16 females; 15.1±2.7 years of education), and 15 were classified with AD (mean±SD age=78.6±6.1 years; 7 females; 16.4±2.26 years of education). All participants were White non-Hispanic. Although most aMCI and AD participants were recruited from a cognitive disorders clinic, clinical status was confirmed with the Alzheimer's Disease Neuroimaging Initiative classification battery, which included the Mini Mental Status Examination, Clinical Dementia Rating Scale, and the Wechsler Memory Scale—Revised Logical Memory II Paragraph A.

Participants were included if they were 65 years of age and had a knowledgeable collateral source available to comment on their cognition and daily functioning. Participants were excluded for medical comorbidities likely to affect cognition (including neurological conditions, current severe depression, substance abuse, and major psychiatric conditions); the inability to complete MRI; the inability to complete cognitive and motor assessments due to inadequate vision, hearing, or manual dexterity; and enrollment in any clinical drug trial related to anti-amyloid agents. Additional exclusion criteria included elevated depression (15-item Geriatric Depression Scale score >5), and moderate or severe dementia (Clinical Dementia Rating score or a Mini Mental Status Examination score <20). This study was approved by the University of Utah Institutional Review Board. All participants provided informed consent as self or by proxy prior to enrollment in accord with the Helsinki Declaration of 1975.

Participants underwent extensive neuropsychological assessment; however, only the Delayed Memory Index from the Repeatable Battery for the Assessment of Neuropsychological Status (RBANS) was examined here for memory function. All subtests were administered and scored as defined in the manual, and normative data from the RBANS manual were used to calculate this Index score as an age-corrected standard score (M=100, SD=15) with higher scores indicating better cognition. Mean±SD RBANS Delayed Memory Index scores were 110.5±9.9, 70.1±18.0, and 50.1±9.9 for the CU, aMCI, and AD groups, respectively, consistent with their clinical status.

Timed Motor Task

Visual demonstration of the motor task can be viewed on Open Science Framework (https://osf.io/phs57/wiki/Functional_reaching_task/), and examples of use are available. This task has also been validated against clinical activities of daily living measures in aMCI patients. To summarize, participants use a standard plastic spoon to acquire two raw kidney beans at a time from a central cup (all cups 9.5 cm diameter and 5.8 cm deep) to one of three distal cups arranged at a radius of 16 cm at −40°, 0°, and 40° relative to the central cup. Participants used their nondominant hand (to avoid ceiling effects), and started by moving to the cup ipsilateral (same side) of the hand used. They then returned to the central cup to acquire two more beans at a time to transport to the middle cup, then the contralateral cup, and then repeated this sequence four more times for a total of 15 out-and-back movements. Task performance was recorded as trial time (in seconds, via stopwatch); lower values indicate better performance. Movement errors, such as dropping beans mid-reach, were recorded; however, only 1% of all reaches had any errors, and the error rate was similar across groups (p=0.70).

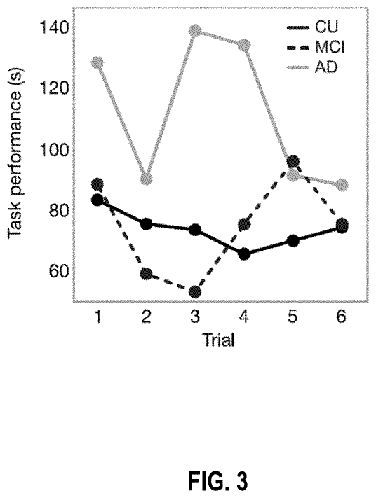

Participants completed 6 trials of the task. This amount of practice was based on a) previous work demonstrating that cognitively intact older adults typically reach stable performance after 5 trials, and b) clinical pragmatism to minimize participant burden (˜5 minutes to administer). Task acquisition was measured as the amount of variability (inter-subject standard deviation) in performance across the practice trials, such that higher standard deviations indicated less task acquisition. FIG. 3 illustrates different degrees of acquisition for an individual CU, aMCI, and AD participant, such that the CU participant had better acquisition (less variability) and the AD participant has worse acquisition (more variability) across trials.

This illustrates the value of administering more than one trial of the task, allowing for any practice effect. Additional measures of performance included overall mean (averaged across the six trials) and acquisition ‘slope’ (Trial 6-Trial 1). The measure of acquisition slope is similar to the California Verbal Learning Test learning slope, which can differentiate between demented and nondemented older adults.

MR Imaging Procedure

Acquisition of imaging data was performed at the Utah Center for Advanced Imaging Research (UCAIR) using a 3.0-T Siemens Prisma scanner with a 64-channel head coil. Structural data was acquired using an MP2RAGE pulse sequence (TR=5000, TE=2.93, acquired sagittally, resolution=1×1×1 mm) to obtain high quality whole-brain 1 mm isotropic T1w images with improved signal homogeneity in ˜7 minutes. Structural MRI scans were processed using FreeSurfer image analysis suite v6.0 (http://surfer.nmr.mgh.harvard.edu/). Technical details are described previously. Left and right hippocampal volumes were adjusted by estimated total intracranial volume (eTIV, cm3) to account for differences in head size, and then summed to yield bilateral hippocampal volumes.

Statistical Analysis

All analyses were performed in R (v3.5.1). Both hippocampal volume and mean task performance were first compared between groups using a one-way ANOVA to determine differences between clinical status. Multivariate linear regression was then conducted to predict bilateral hippocampal volume using participants' motor task acquisition (i.e., standard deviation) as a predictor while controlling for age, sex, years of education, clinical status, and RBANS Delayed Memory Index score; these factors were included given their known associations with hippocampal volume. The normality assumption for bilateral hippocampal volume was tested using the Shapiro-Wilk test. The motor task variables of acquisition, overall mean, and acquisition slope were separately added to the null regression model (age, sex, years of education, clinical status, and RBANS Delayed Memory Index score) to determine if the contribution of the motor task provided additional predictive value beyond the RBANS Delayed Memory Index score. Akaike's Information Criteria (AIC) and adjusted R2 values from each model were compared.

### Results

One-way ANOVA confirmed a significant effect of clinical status on bilateral hippocampal volumes (F_{2,53}=15.6; p<0.0001) (CU=4.55±0.79 cm3, 95% CI [4.11, 4.98]; aMCI=3.56±0.55 cm3, 95% CI [3.33, 3.80]; AD=3.16±0.89 cm3, 95% CI [2.67, 3.65]), consistent with their diagnosis. Furthermore, data showed that this motor task was feasible even for participants with mild AD, even though there was main effect of group on mean task performance (F_{2,53}=5.52; p=0.007) with the AD group as the slowest. Mean task performance was not significantly related to hippocampal volume (p=0.14), after controlling for age, sex, education, clinical status, and memory score. As shown in the individual participant data in FIG. 3, the AD participant was not only slower but also had greater variability across trials (i.e., less task acquisition). Across participants, this variability across trials was sensitive to hippocampal volume, given that regression analyses revealed that motor task acquisition (measured as standard deviation of performance across the 6 practice trials) was a significant predictor of bilateral hippocampal volume (β=−0.03, 95% CI [−0.07, −0.0009]; p=0.04), even when controlling for age (p=0.46), sex (p=0.50), education (p=0.33), clinical status (p=0.25), and memory score (β=0.01, 95% CI [−0.003, 0.02], p=0.11). The full model yielded an adjusted R2=0.36 (F4,47=6.1; p<0.0001). Comparison of the linear regression models demonstrated that adding either the motor acquisition or mean motor task performance variable to the null model yielded incremental improvements in predicting hippocampal volume, while adding acquisition slope increased the percent variance explained in hippocampal volume by 9% compared to the RBANS Delayed Memory Index (Table 1).

TABLE 1
Linear regression results for models with each motor task variable
as a predictor of hippocampal volume, compared to the null model
including RBANS Delayed Memory Index. Age, sex, years of education,
and clinical status are controlled for in each model.

| Model | β | 95% CI | | p-value | R² | AIC |
|---|---|---|---|---|---|---|
| RBANS Delayed Memory Index | 0.009 | (−0.005, | 0.02) | 0.21 | 0.32 | 128 |
| Motor task acquisition (SD) | −0.03 | (−0.06, | −0.009) | 0.04 | 0.36 | 125 |
| Mean motor task performance | −0.008 | (−0.02, | 0.003) | 0.14 | 0.34 | 127 |
| Acquisition slope (Trial 6-Trial 1) | 0.02 | (0.004, | 0.03) | 0.007 | 0.41 | 121 |

This study tested the relationship between bilateral hippocampal volume and acquisition of a motor task in cognitively unimpaired, aMCI, and mild AD older adults. Results showed that even after controlling for age, gender, education, clinical status, and memory, motor task acquisition was still a significant predictor of hippocampal volume, with worse task acquisition (i.e., more variable performance) being associated with lower hippocampal volume. This suggests that motor practice effects may better indicate hippocampal atrophy even after controlling for other clinical factors, which is particularly relevant for cases of MRI contraindication.

Although several complex upper-limb tasks have been shown to be sensitive to disease severity, this is among the first to associate motor behavior with an AD biomarker. This work highlights the value of evaluating multiple trials of a motor task, rather than a “one-and-done” approach in which a single attempt could mask relevant differences. This is consistent with extensive work showing the clinical utility of cognitive practice effects. Furthermore, these findings are consistent with behavioral data linking practice effects on this motor task with visuospatial scores, suggesting a potential mechanism underlying the relationship to hippocampal volume shown here. Without being bound by theory, declines in motor acquisition will track with hippocampal atrophy (or other biomarkers) over time.

Without begin bound by theory, future research in larger and diverse cohorts will reveal that the presently disclosed subject matter can serve as an affordable enrichment strategy for AD clinical trials. We also acknowledge that this study does not directly compare this motor task to other existing motor tasks (e.g., grip dynamometry, 10-Meter Walk Test), although we have previously shown that the motor task presented here is more sensitive to disease severity.

In addition to the growing evidence for this motor task as a relevant measure for AD, it should be highlighted that only ˜5 minutes are needed to administer several trials, and the apparatus costs <$10 to fabricate from household items, thereby improving detection of hippocampal atrophy with virtually no additional time or cost. It is also extremely portable, making it easy to administer outside of a clinical setting (e.g., at a community center or at home). Eliminating the need for medical staff/settings has the potential to better serve under-represented minorities. Future studies will test the reliability of administering this motor task in various settings across a more diverse sample.

### Example 4

Improving Prediction of Amyloid Deposition in Mild Cognitive Impairment with a Timed Motor Task

Abstract

Cortical amyloid deposition is one of the hallmark biomarkers of Alzheimer's disease. However, given how cost- and time-intensive amyloid imaging can be, there is a continued need for a low-cost, non-invasive, and accessible enrichment strategy to pre-screen individuals for their likelihood of amyloid prior to imaging. Previous work supports the use of coordinated limb movement as a screening tool, even after controlling for cognitive and daily function. Thirty-six patients diagnosed with amnestic Mild Cognitive Impairment over the age of 65 underwent F-Flutemetamol amyloid-positron emission tomography imaging, then completed a timed motor task involving upper limb coordination. This task takes ˜5 minutes to administer and score. Multivariate linear regression and Receiver Operator Characteristic analyses showed that including motor task performance improved model prediction of amyloid burden. Results support the rationale for including functional upper extremity motor assessment as a cost- and time-effective means to screen participants for amyloid deposition.

Introduction

Cortical amyloid deposition is one of the hallmark biomarkers of Alzheimer's disease (AD) and its progression. Thus, numerous large-scale clinical trials in preclinical AD have focused on therapies aimed at clearing beta-amyloid neuritic plaques to slow disease progression. However, recruiting and enrolling asymptomatic individuals who are amyloid positive is time-consuming, since only ˜30% of cognitively-intact individuals have elevated levels of amyloid. This means that two out of every three individuals who undergo amyloid positron emission tomography (PET) as part of the screening process for clinical trial recruitment will not be eligible for enrollment. Furthermore, amyloid imaging is expensive, exposes individuals to radiation, and can only be completed select sites with the necessary technology and expertise. Thus, there is a need for a low-cost, non-invasive, and accessible way to pre-screen asymptomatic individuals for their likelihood of β-amyloid neuritic plaque density prior to PET imaging.

Although complex movements involving multi-limb coordination have been associated with disease severity, recent work has also demonstrated that such movement may be sensitive to disease progression when assessed with a timed motor task. To minimize cost and assessment time and improve portability, we developed an upper extremity motor task that i) does not require any hardware or software; can differentiate between cognitively intact and cognitively impaired individuals better than other simple motor tasks (i.e., grip strength, see); and iii) is feasible for amnestic Mild Cognitive Impairment (MCI) cohorts. This is in contrast to other assessments of complex movement that require demanding technology (e.g., movement sensors, motion capture technology, electromyography, or transcranial magnetic stimulation) or do not show strong prognostic effects at baseline. Given the relative advantages of this timed motor task and its prediction of functional decline in MCI, we hypothesized that task performance would be related to the extent of amyloid plaque deposition, and would improve the classification of amyloid positivity in individuals with amnestic MCI, above and beyond baseline cognitive and activities of daily living.

Materials & Methods

Participants: Thirty-six participants with amnestic MCI from a larger clinical trial sample (ClinicalTrials.gov Identifier: NCT02301546; currently active, not recruiting) participated (mean±SD age=73.25±5.5 years; 13 females; 16.81±3.0 years of education; 97% white). Inclusion criteria were 65 years old or older, had a collateral source available to answer questions about thinking abilities and daily activities, had access and the ability to use a computer and the internet, spoke English, and demonstrated that they had single- or multi-domain amnestic MCI. MCI was categorized as: 1) concern of a change in cognition from the participants or a knowledgeable informant, 2) impairment in memory (and other cognitive domains), with at least one cognitive test score in a domain being 1.5 standard deviations below an estimate of premorbid intellect, and 3) independence of daily functioning. Exclusion criteria were history of major neurological (e.g., stroke, Parkinson's disease) or psychiatric illnesses (e.g., schizophrenia, bipolar disorder) or substance abuse, current major depression (>7 on the 15-item Geriatric Depression Scale), or cognitive impairment suggestive of dementia. This study was approved by the University of Utah Institutional Review Board, in accordance with the World Medical Association Declaration of Helsinki. All participants provided informed consent as self or by proxy prior to enrollment.

Timed motor task: A full visual description of the timed motor task can be viewed on Open Science Framework (https://osf.io/phs57/wiki/Functional_reaching_task/), and certain examples of use are available. To summarize, participants use a standard plastic spoon to acquire two raw kidney beans at a time from a central cup (all cups 9.5 cm diameter and 5.8 cm deep) to one of three distal cups arranged at a radius of 16 cm at −40°, 0°, and 40° relative to the central cup. All cups were the same size. Participants were tested using their nondominant hand, and started by moving to the cup ipsilateral (same side) of the hand used. They then returned to the central cup to acquire two more beans at a time to transport to the middle cup, then the contralateral cup, and then repeated this sequence four more times for a total of 15 out-and-back movements. Task performance was measured as trial time (in seconds), i.e., how long it took to complete 15 movements, such that lower values indicate better performance. Movement errors, such as dropping beans mid-reach, were recorded; however, only 1 error (0.1% of all reaches) was made in this dataset. Participants first completed 3 trials for practice and task familiarization.

Amyloid-PET imaging: Participants received F-Flutemetamol imaging as described previously. F-Flutemetamol was produced under PET cGMP standards and the studies were conducted under an approved Federal Drug Administration Investigational New Drug application. Imaging was performed 90 minutes after the injection of 185 mBq (5 mCi) of F-Flutemetamol. Emission imaging time was approximately 20 minutes. A GE Discovery PET/CT 710 (GE Healthcare) was used in this study. This PET/CT scanner has a full width at half-maximum spatial resolution of 5.0 mm and excellent performance characteristics. F-Flutemetamol uptake was analyzed using a regional semi-quantitative technique. In this technique, semi-quantitative regional (prefrontal, anterior cingulate, precuneus/posterior cingulate, parietal, mesial temporal, lateral temporal, occipital, sensorimotor, cerebellar grey matter, and whole cerebellum) regional standardized uptake value ratios (SUVR) were generated automatically and normalized to the pons. Based on the regional values a composite standardized uptake value ratio (composite SUVR) of the cerebral cortex was generated automatically and normalized to the pons using the CortexID Suite software. This software uses a threshold z score of 2.0 to indicate abnormally increased regional amyloid burden that corresponds to a composite SUVR of 0.59 when normalized to the pons, providing a 99.4% concordance with visual assessment. For F-Flutemetamol amyloid imaging, there is no specific age-related “normal” level of binding in the CortexID Suite database to assess age-matched normality. Thus, the study images were compared to the intrinsic software database control group as a whole to calculate the z-scores compared to clinically negative amyloid scans.

Measures of cognitive and daily functioning: As part of the clinical trial, participants underwent extensive neuropsychological assessment at baseline; however, only the Delayed Memory Index from the Repeatable Battery for the Assessment of Neuropsychological Status (RBANS) was examined here. All subtests were administered and scored as defined in the manual, and normative data from RBANS manual was used to calculate the Index score, which are presented as age-corrected standard score (M=100, SD=15) with higher scores indicating better cognition. Mean±SD RBANS Delayed Memory Index scores for this sample were 74.42±21.01, consistent with their diagnosis. Baseline activities of daily living (ADL) function was measured using the self-report portion of the 18-item Alzheimer's Disease Cooperative Study-Activities of Daily Living scale adapted for MCI (ADCS-ADL-MCI). Possible scores on this scale range from 0 to 57, with higher scores indicating better daily functioning. Mean±SD ADCS-ADL-MCI scores were 46.08□3.82, again consistent with their diagnosis.

Statistical analysis: Multivariate linear regression was conducted to predict F-Flutemetamol pons normalized composite SUVRs using participants' motor task performance (i.e., trial time) as a predictor while controlling for age, gender, years of education, RBANS Delayed Memory Index score, and ADCS-ADL-MCI-18 score. Assumptions for regression were inspected visually using Q-Q plots and all analysis were performed in R (v3.5.1). Statistical models with and without motor task performance as a dependent variable were compared by analysis of variance to determine if the contribution of motor task performance to prediction accuracy was statistically significant.

To test whether motor task performance improved amyloid positivity classification (Aβ+ or Aβ−), we first developed a null model using best practices of model selection that included age, sex, education, RBANS Delayed Memory Index score, and ADCS-ADL-MCI-18 score. A generalized linear model was selected since amyloid positivity follows a binomial distribution. We then generated a motor task model that included the null model plus the motor task variable. Akaike information criteria (AIC) and analysis of variance (ANOVA) using a Chi-squared distribution were used to test for model superiority (null vs. task). This determined if including motor task performance as a variable improved prediction accuracy of amyloid classification without added model complexity. An AIC difference of >3 between the null and task model would indicate improved data fit by the task model. Receiver operator characteristics (ROC) and precision recall curves were also generated to assess model specificity, sensitivity, precision and recall with and without motor task performance.

Results

No adverse events were reported during the injection, uptake time, or imaging studies with the investigational imaging agent F-Flutemetamol. Mean composite of SUVRs normalized to the pons was 0.68 (SD=0.18, range=0.41-0.97). Mean motor task performance was 63.88 seconds (SD=15.66, range=39.81-121.75). For reference, cognitively-intact older adults tend to be faster (M=58.50 seconds, data from).

Regression analyses revealed that motor task performance was a significant predictor of composite SUVR (13=0.004; 95% CI=[0.0004, 0.008]; p=0.03), even when controlling for age (p=0.17), gender (p=0.1), years of education (13=0.03; 95% CI=[0.013, 0.05]; p=0.002), RBANS Delayed Memory Index score (p=0.34), and ADCS-ADL-MCI score (p=0.25). The full model yielded an adjusted R²=0.25 (F(6,29)=3.11; p=0.022). Comparison of regression models with and without motor task performance (R²=0.15, p=0.08) through analysis of variance demonstrated that the inclusion of motor task performance significantly improved prediction (p=0.03) of composite SUVR by over 65%.

Based on established thresholds, 26 of the 36 participants (72%) were classified as amyloid-positive. The best generalized linear model of the covariate data, i.e. the null model, included age, sex, education, RBANS Delayed Memory Index score, and ADCS-ADL-MCI score (AIC=44.1) as predictors of amyloid positivity classification. Adding motor task performance to the null model improved model accuracy (AIC=41.4). ANOVA confirmed that the motor task model was more accurate than the null model (p=0.03) in predicting amyloid classification.

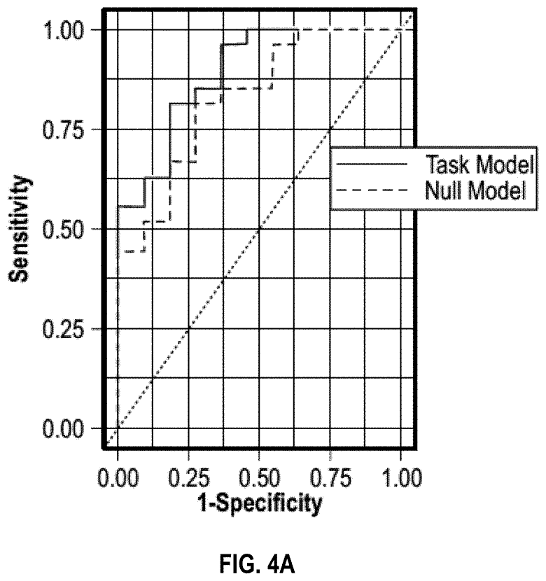

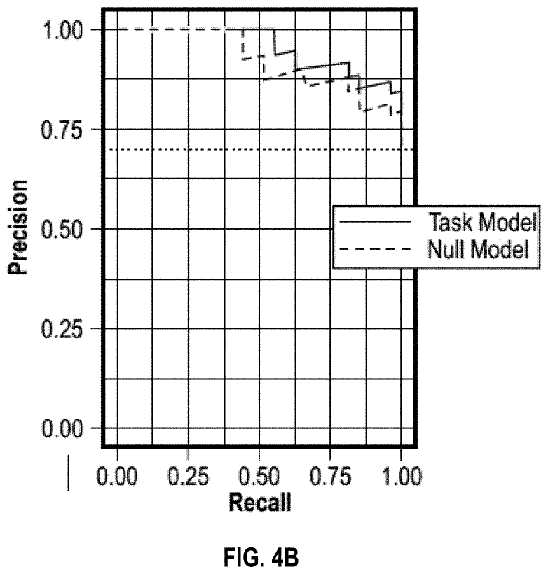

ROC showed that the motor task model had a specificity of 60% (6/10 prediction accuracy of Aβ−), and a sensitivity of 88% (23/26 prediction accuracy of Aβ+) with an overall accuracy of 75% compared to the null model, which had a specificity of 50% (5/10 prediction accuracy of Aβ−) and a sensitivity of 93% (24/26 prediction accuracy of Aβ+) with an overall accuracy of 80%. Overall, the motor task model had an AUC of 90%, compared to the null model AUC of 84% (FIG. 4A).

Given that the majority of participants were classified as amyloid positive, precision recall curves (PRC) were also generated for each model. Briefly, a precision recall curve determines the trade-off of a model between its true-positive rate and its positive prediction rate by varying the ratio between positive and negative cases and assessing the predictive skill of the model throughout. This can be an especially important metric when evaluating samples with a disproportionate number of positive or negative cases. Here, the area under the PRC of the motor task model was 96% compared to that of the null model, which was 93% (FIG. 4B This further demonstrates that advantage of including motor task performance for predicting amyloid-positive cases even when the ratio between positive and negative cases may be skewed, such as in preventative clinical trials where the number of amyloid-negative cases is much higher (e.g., 25).

To determine an optimal cut-off of motor task performance to predict amyloid-positive cases, a permutation test was run that varied motor task cut-off threshold across the range of performance times observed in this sample, followed by a calculation of the resulting odds ratio for amyloid positivity. The cut-off value with the highest odds ratio was determined to be the optimal threshold, which was a task performance of 68 seconds with an odds ratio of 4.76. Thus, data from subjects diagnosed with amnestic Mild Cognitive Impairment show that after three practice trials, a performance of about 68 seconds or more was associated with an odds ratio of about 4.76 for being amyloid-positive. This threshold suggests that a person with a task performance greater than about 68 seconds on their fourth trial would be nearly five times more likely to be amyloid positive than a person with a motor task performance below 68 seconds.

Discussion

The purpose of this brief report was to test whether performance on a timed motor task was related to the extent of amyloid plaque deposition in individuals with amnestic MCI, and would improve the classification of amyloid positivity. Results showed that even after controlling for age, gender, education, delayed memory, and ADL function, motor task performance was still a significant predictor of composite SUVR, with worse task performance being associated with more amyloid deposition. Furthermore, adding motor task performance as a predictor variable improved amyloid positivity classification, being able to better identify individuals with elevated amyloid than with just age, gender, education, delayed memory, and ADL function. Overall, these findings support the rationale for including functional upper extremity motor assessment as a means to better screen participants for clinical trial recruitment that requires elevated amyloid for enrollment (e.g., Anti-Amyloid Treatment in Asymptomatic Alzheimer's Disease [A4]).

Although several complex upper extremity motor tasks have been shown to be sensitive to disease severity, this is among the first to show a relationship with disease biomarkers, above and beyond other measures such as memory or ADL function. While this study does not provide a clear mechanism of this relationship, it possible that unimanual motor performance may be sensitive to amyloid deposition patterns in sensory-motor areas specifically, which may track with global composite measures. It is also likely that this task, more so than grip dynamometry or finger tapping that do not have a strong visuospatial demand, recruits relevant neural structures (e.g., hippocampus) that are particularly susceptible to early stages of dementia. Future research is needed, however, to further explore the underlying mechanism between complex motor tasks and both global and regional amyloid deposition.

It is acknowledged that screening for amyloid deposition is already a time- and cost-intensive process, particularly in mild cases or those who are asymptomatic. Efforts to identify Aβ+ individuals have been enriched by additional biomarkers, genetic testing, and extensive neuropsychological evaluation, which also take time and/or money, and are still not always sensitive and specific to amyloid or disease progression. We therefore highlight the fact that the motor task used in this study takes <5 minutes to administer and costs less than $10 to fabricate from household items, thereby improving the likelihood of identifying individuals with amyloid accumulation with virtually no additional time or cost. It is also extremely portable, with data collection easily available in clinics and the community. In fact, using these time and cost parameters as inputs into the Biomarker Prognostic Enrichment Tool (BioPET), along with published rates of amyloid positivity in cognitively-intact adults, it is estimated (with a power of 0.9) that just by pre-screening individuals with the timed motor task could reduce the total cost for amyloid scanning by ˜36%. For example, in a preventative AD clinical trial that attempts to recruit 1,000 amyloid-positive subjects, this 36% could reflect millions of dollars in savings (as well as countless hours for the study personnel and patients and their families).

Furthermore, the task's extremely low price and rapid testing time compared to amyloid-PET still outweigh the estimated 1.5×increase in total individuals screened, thereby streamlining and improving the efficiency of clinical trial recruitment through additional enrichment strategies.

Without begin bound by theory, the presently disclosed subject matter provides as an affordable enrichment strategy for AD clinical trials. Further, the motor task and subject matter disclosed herein is likely more sensitive to disease severity than other motor assessments. As such, motor assessments have promise as cost-effective and non-invasive screening tools that would allow for enriching samples in clinical trials in AD.

### Example 5

SUMMARY: Many current clinical trials in Mild Cognitive Impairment and Alzheimer's disease are using biomarkers (e.g., “positive” on amyloid imaging) as part of the inclusion criteria. Similarly, biomarkers are becoming increasingly important in the diagnosis of Alzheimer's disease and other types of dementia. However, many of these biomarkers are costly, invasive, provide little clinical information, and may be only completed at sites with unique resources. The rationale for this project is the need for more practical markers of disease and its progression, which could be used to enrich clinical trials. Research suggests that motor behavior is a simple and widely accessible solution for improving enrichment and streamlining participant screening that has substantial cost- and time-savings. The long-term goal is to develop a quick, cheap, and easy-to-administer motor assessment as a tool for prognostic enrichment of AD clinical trial selection or for making AD screening more accessible. The overall objective of this new application is to demonstrate that individuals with poorer motor task acquisition are likely to be identified as “positive” on amyloid imaging and other biomarkers. This project leverages existing cohorts of older individuals who are cognitively intact as well as those with Mild Cognitive Impairment and Alzheimer's disease. The aims of this project will offer more efficient screening in recruiting for clinical trials, which would reduce participant burden and financial costs associated with these trials. The disclosed motor task could also be used to enrich trials with those more likely to progress and to monitor treatment benefit as a proximal outcome measure. The affordability, portability, and accessibility of the disclosed motor task are also relevant to Goal F of the National Institute on Aging's Strategic Directions for Research, 2020-2025 by offering a tool that could address three key health disparities: socioeconomic status, geographic location, and health care access.

This disclosure is relevant to public health because the apparatus and methods disclosed herein uses human movement to develop an affordable and accessible way to predict amyloid positivity in older adults with and without cognitive impairment (as well as other Alzheimer's disease biomarkers, like atrophic hippocampi and APOE e4). There is potential for a significant advance in the cost and inclusivity of AD research and treatment. Thus, present disclosure is relevant to the National Institute on Aging's recent interest to study motor system changes as a predictor of preclinical Alzheimer's disease.

### Using a Rapid Motor Task to Enrich Clinical Trials in Alzheimer's Disease Requiring Amyloid Positivity

Without being bound by theory, this disclosure demonstrates the utility of motor acquisition (i.e., improved task performance due to practice) as an inexpensive, non-invasive, and widely available tool for screening individuals for therapeutic clinical trials requiring amyloid positivity. This approach could significantly reduce participant burden and financial costs of these clinical trials by streamlining recruitment. Utilizing this motor learning tool can also make trials safer, by limiting radiation exposure in subjects unlikely to have abnormally high amyloid accumulation. These benefits are most realized in prevention trials in pre-symptomatic Alzheimer's disease (AD) by enriching samples with individuals most likely to meet the inclusion criteria of amyloid positivity and progression across time. For example, the landmark Anti-Amyloid Treatment in Asymptomatic Alzheimer's Disease (A4) trial enrolled 1,169 participants with elevated levels of brain amyloid plaque. However, over 4,000 individuals were screened but did not meet the amyloid burden criteria. This clearly illustrates the need for rapid, affordable enrichment strategies that will improve screening decisions while saving time and resources. There is new interest in using the motor system to predict preclinical AD. In embodiments, the present disclosure employs motor learning to predict amyloid burden and accelerated functional decline in amnestic Mild Cognitive Impairment (aMCI). We have developed a rapid (˜5 minutes), affordable (<$10), and fully portable (even mailable) motor task that involves upper extremity movement. We have shown that task acquisition correlates with other AD biomarkers, is more sensitive to cognitive impairment than other motor tasks, and is feasible for aMCI and early AD patients. These features are a clear advantage compared to other motor assessments that do not track with AD biomarkers, fail to predict prognosis, or require demanding technology and data analytics.

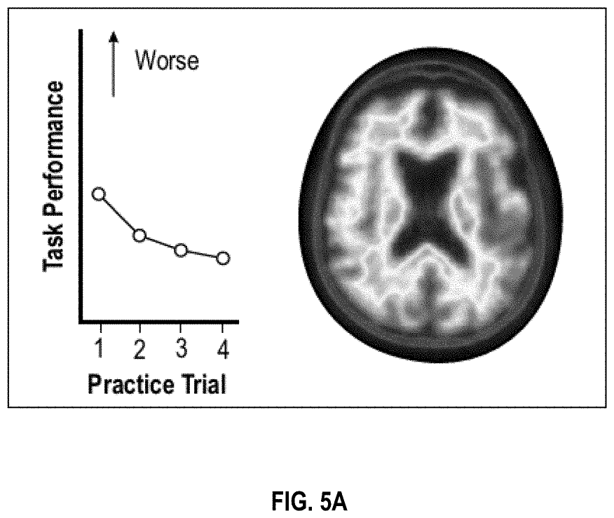

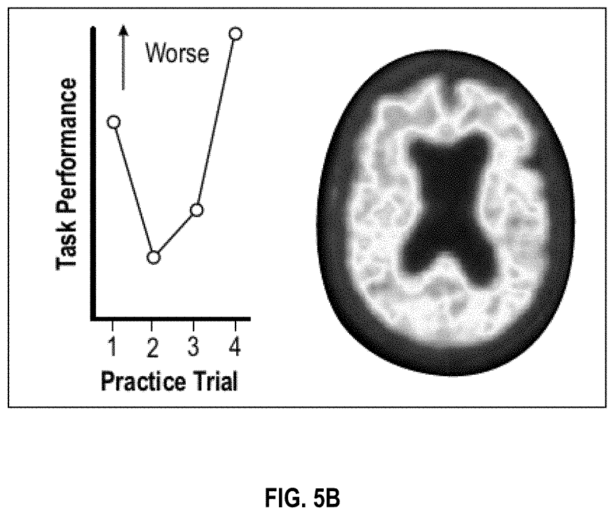

To illustrate the ‘added value’ of our motor task, consider two cases with severely impaired memory. As shown in FIG. 5 Case A had good task acquisition and low amyloid deposition. Conversely, Case B had poor acquisition and high amyloid burden. In 36 patients diagnosed with aMCI (a prodrome of AD), poor acquisition and high amyloid deposition had an odds ratio of 4.65. Data from our ongoing Amyloid Positivity using Practice Effects (APPE) longitudinal study also suggest that acquisition predicts AD conversion over one year.

Without being bound by theory, individuals with poor task acquisition (i.e., minimal improvement with practice) are more likely to be amyloid-positive, and can also be positive for other relevant AD biomarkers, such as hippocampal volume and cognitive scores. We can leverage our existing longitudinal APPE cohort of cognitively-intact, aMCI, and AD older adults to test the following aims. This will, in turn, offer more economical and efficient screening of potential participants for clinical trials.

The specific aims to be achieved in this project are:
1. Examine the relationship between motor task acquisition and relevant AD biomarkers, cognitive status, and functional status. Poorer acquisition will be correlated with greater β-amyloid neuritic plaque density in the brain ([18F]flutemetamol PET), as well as other established biomarkers (APOE e4 status, hippocampal volume), even after controlling for baseline cognition. Poorer acquisition will also be associated with worse cognition and daily function.
2. Compare changes in motor task acquisition over one year. Compared to cognitively intact older adults, individuals with baseline diagnoses of aMCI and mild AD will show declines in acquisition (i.e., less improvement with practice one year later).
3. Evaluate the ability of motor task acquisition to predict disease conversion. Acquisition cutoffs will also be calculated for identifying participants who will convert to MCI or to AD, defined by ADNI criteria.

Without being bound by theory, we disclose an inexpensive, non-invasive, and widely available screening tool for predicting amyloid positivity and disease progression. The present disclosure can save significant time and resources in future clinical trials in MCI and AD.

Increasingly, therapeutic clinical trials in the spectrum of Alzheimer's disease (AD) and age-related cognitive decline are requiring participants to present with evidence of biomarker positivity before enrollment. This is particularly true for prevention trials. For example, in the Anti-Amyloid Treatment in Asymptomatic Alzheimer's Disease (A4) trial, 4,486 participants were screened to identify 1,323 amyloid positive individuals. Similar clinical trials requiring amyloid positivity as an inclusion criterion are ongoing from multiple pharmaceutical companies (e.g., Lilly, Merck, Roche, Biogen). Such trials are expensive, slow, burdensome to patients and research teams, and expose subjects to unnecessary radiation. Since it will be unsustainable to discard nearly 70% of screened participants, better screening methods are needed for identifying subjects likely to be amyloid biomarker positive. In 2012, the Food and Drug Administration (FDA) provided guidance for industry-sponsored clinical trials on using enrichment strategies. Such strategies could better select study populations in which detection of a drug effect (if there is one) is more likely than in an unselected population. In addition to reducing noise within the target population, the FDA recommended choosing patients with a greater risk of worsening (prognostic enrichment) and those most likely to respond to the intervention (predictive enrichment). To date, clinical trials in Mild Cognitive Impairment (MCI) and AD have struggled to identify potent enrichment variables that could be used on a large scale. Existing enrichment biomarkers in AD (e.g., β-amyloid neuritic plaque density via PET imaging, tau in cerebrospinal fluid via lumbar puncture and PET imaging, brain metabolism via FDG-PET imaging, hippocampal volumes via MRI, Apolipoprotein E e4 via blood draw) are expensive, invasive, and/or expose potential participants to unnecessary risks. Additionally, many of these enrichment variables identify individuals at greater risk of progression (e.g., MCI converting to AD) rather than individuals at greater likelihood of biomarker positivity.

A proposed solution: In contrast to existing AD biomarkers, measuring voluntary movement can be extremely easy, quick, cheap, and safe. Established motor measures, however, have lacked specificity to the progression of AD. For example, gait speed and grip strength are both sensitive to cognitive status, but these measures also decline with normal aging and therefore may not be specific to AD. Existing motor measures also have not shown strong associations with current AD biomarkers or risk factors (e.g., β-amyloid deposition, hippocampal atrophy, Apolipoprotein E (APOE)). Other more experimental tasks, like finger tapping do not correlate with daily functioning, and are therefore not sensitive to disease progression specifically. However, as evidenced by the Notice of Special Interest (NOT-AG-20-053): “Sensory and Motor System Changes as Predictors of Preclinical Alzheimer's Disease,” there is renewed interest in investigating motor systems as they relate to preclinical AD. Our work directly addresses this Notice. Briefly, the presently disclosed motor task (1) is sensitive to cognitive status; (2) is a better predictor of one-year functional decline than cognitive testing; (3) correlates with hippocampal volume; and improves classification of amyloid-positive cases. Specifically, we have developed a motor measure that is more sensitive to cognitive status than grip strength in community dwelling older adults. The presently disclosed task involves functional upper-limb movement in which participants use a spoon to acquire and transport raw kidney beans to one of three small plastic cups in a simple sequence (left, middle, right) as quickly yet as accurately as possible. Participants are timed with a stopwatch, with faster times indicating better performance. We have shown that this task predicts one-year decline in both subjective and objective measures of daily functioning in patients with amnestic MCI, as well as amyloid positivity and hippocampal volume. Our preliminary data suggest that it predicts one-year conversion to AD. We note that out motor task outperforms cognitive scores in predicting disease progression. These findings in and of themselves are very promising, especially as the FDA continues to encourage function (in addition to cognition) as the therapeutic endpoint for AD drug trials. Thus, our motor task could be used to enrich clinical trials for individuals who are expected to show more functional decline.

Our motor task could also enrich clinical trials for biomarker positivity. For example, we have shown that it is significantly related to hippocampal volume across the AD spectrum (cognitively intact vs. amnestic MCI vs. AD), even after controlling for age, sex, and memory score. Without being bound by theory, the presently disclosed motor task and apparatuses associated therewith will enrich clinical trials by screening for individuals who are amyloid-positive. Our data show an odds ratio of 4.76 for individuals who were slower than 68 seconds on the task by their 4th practice trial. This performance threshold also reflects a practice or learning effect in our task, such that elevated amyloid may affect how the task is acquired over three to four practice trials of the task rather than performed in just a single or averaged score. This is illustrated below in Cases A and B, who are matched for age, sex, education, and memory (FIG. 5). Case A (top) is amyloid-negative (based on PET) and shows good motor acquisition as she improves across the four trials of the motor task, ending with a score of 63 seconds. In contrast, Case B is amyloid-positive and has poor acquisition (initial improvement but then worsening across trials), ending with a motor task score of 94 seconds. Importantly, performance on the first trial was somewhat comparable between the cases, which highlights the value of evaluating multiple trials of a motor task, rather than a “one-and-done” approach in which a single attempt (or an average) could mask relevant differences in biomarkers or clinical events. High intra-subject variability in finger tapping has been also been correlated with amyloid levels in cerebral spinal fluid. Our finding is consistent with extensive work showing the clinical utility of cognitive practice effects, and will complement the work already being done in the longitudinal “Enriching Clinical Trials Requiring Amyloid Positivity with Practice Effects (APPE)” cohort (R01AG045163; PI: Duff) that is be leveraged here.

The presently disclosed systems and methods are inexpensive and brief. Screening for amyloid deposition is already a time- and cost-intensive process. Efforts to identify amyloid-positive individuals have been enriched by additional biomarkers, genetic testing, and extensive neuropsychological evaluation, all of which expend valuable resources for potential participants and researchers, yet are not always sensitive and specific to amyloid or disease progression. In contrast, only a stopwatch and the task apparatus (beans, spoon, and cups) is needed to measure performance on this task, and collecting four trials of this motor task takes ˜5 minutes. Embodiments of the task apparatus itself costs less than $10 to fabricate. Thus, without being bound by theory, the present disclosure will enrich patient samples for elevated amyloid, hippocampal atrophy, or disease progression at virtually no additional time or cost.

While other technologies may provide precise measurement of motor behavior, they can be costly and time consuming to calibrate, and therefore not realistic for large-scale screening in community samples. Meanwhile, the presently disclosed motor task is extremely portable, making data collection fast and easy in clinics and the community.

Using this Motor Task can Save Time and Money in ADRD Clinical Trials.

Using the time and cost parameters (5 minutes and $10) as inputs into the Biomarker Prognostic Enrichment Tool (BioPET), along with published rates of amyloid positivity in cognitively-intact adults, we estimate with a power of 0.9 that just by pre-screening individuals with our motor task could reduce the total cost for amyloid scanning by ˜36%. Thus, in a hypothetical preventative AD clinical trial attempting to recruit 1,000 amyloid-positive subjects, this 36% could reflect millions of dollars in savings (as well as countless hours for the study personnel and patients and their families). Furthermore, the task's extremely low price and rapid testing time compared to amyloid-PET still outweigh the estimated 1.5×increase in total individuals screened, thereby streamlining and improving the efficiency of clinical trial recruitment through additional enrichment strategies.

As further described in ‘Innovation’, our motor task can be used as a pre-screening tool for anti-amyloid treatment clinical trials. Pharmaceutical companies and researchers could utilize this task remotely by mailing it to interested participants to complete in their own homes, then using the cost savings to fly and house a streamlined (and more diverse) subset of potentially eligible participants to the imaging center for further screening.

This disclosure challenges and seeks to shift current research and clinical paradigms in multiple ways.

1. It introduces a new motor task. A number of upper- and lower-extremity motor tasks have been proposed in the context of AD, such as grip strength, finger tapping, gait speed, dissociated hand and eye movement, alternating flexion and extension of the arm, and dual task conditions of arm movement or gait. While all of these proposed measures appear to be sensitive to clinical status (e.g., performance is worse in cognitively impaired adults than intact adults), their relationship to relevant biomarkers across the AD spectrum can be limited. Grip strength is one of the most commonly used motor measures in aging research, but when compared side-by-side using Receiver operating characteristic (ROC) curves, our motor task is more sensitive and specific to global cognition than grip strength with an AUC of 0.71 compared to 0.59, respectively. Our task also shows no effect of sex, giving it an additional advantage over grip strength and other manual muscle testing. Moreover, the lack of association with amyloid makes grip strength a poor enrichment candidate, despite its ease of administration. We have also shown that education level does not affect our task, making it advantageous over cognitive testing, which consistently shows educational bias. Thus, this proposal offers a scientifically rigorous and less biased enrichment strategy that is affordable, easy to administer in a number of settings, and compared to multiple AD biomarkers.

2. We show the value of measuring change in motor performance over multiple attempts, rather than collecting a single trial or averaging across several trials. Our data show a stronger link between motor task acquisition across trials and AD biomarkers than the average or first-trial performance of our task. Our motor task also avoids ceiling or floor effects, which occur when other motor measures are repeatedly collected (e.g., MDS-UPDRS, gait speed, finger tapping), preventing any learning effect to be observed.

This project compares our motor task to multiple AD biomarkers. There are currently very few studies that correlate such biomarkers with motor tasks (like amyloid-PET and grip strength or hand movement on a smartphone app), and more often in animal models (e.g., rotarod in mice). While there is a strong premise to using motor tasks in conjunction with other AD biomarkers (see ‘SIGNIFICANCE’ as well as NOT-AG-20-053 itself), few have been compared across a range of biomarkers side-by-side across the AD spectrum, as is disclosed here.

This technology disclosed herein offers an objective measure of an activity of daily living. Whereas most measures of daily functioning in observational studies and clinical trials are subjective reports of the patient and/or collateral (e.g., Functional Assessment Questionnaire (FAQ), Clinical Dementia Rating (CDR) scale), our motor task yields a performance-based score that is tied to daily functioning. Furthermore, our task is scored as a continuous variable, rather than an ordinal score in the FAQ and CDR, which makes it ideal for measuring change over time and comparing between groups. Without being bound by theory, we will have extensive data on how motor task performance changes (or not) over time depending on disease stage.

5. Our motor task can be rapidly administered anywhere and does not require clinical supervision, making it accessible and inclusive. We recently conducted a pilot study in which we mailed disposable kits containing our motor task to participants in 33 states for <$10/person. We found that individuals ages 40-80 can reliably administer the task themselves at home and unsupervised, and did so on both weekdays and weekends at all hours. As such, we collected data in this pilot study from 177 participants in 174 days during the COVID-19 pandemic. Theoretically, this task could be sent to anyone with a mailing address (internet not necessary), which could drastically improve the diversity, inclusion, and equity of AD research and patient care. Furthermore, since our task is procedural in nature, it does not require strong literacy nor English language competency, which likely explains the lack of education effect as noted above in point #1. We intentionally designed it with common household items (e.g., spoon, cups) to minimize cultural differences, making it highly accessible and inclusive.

### Example Approach

Without being bound by theory, (i) motor acquisition is related to β-amyloid neuritic plaque density, which can be shown utilizing [18F]Flutemetamol PET imaging and other AD biomarkers in older adults, ii) motor acquisition changes over time across the spectrum of AD, and iii) motor acquisition predicts AD progression. We can leverage the existing APPE longitudinal cohort to collect and analyze motor acquisition data in three cohorts (cognitively intact, amnestic MCI, and probable AD).

### Data

Key features and feasibility of the motor task have been established. We have developed and validated a motor task that involves goal-directed reaching and grasping using everyday household items. Exemplary features of this task are provided below:
Costs <$10
Takes 5 minutes to administer and score.
Does not require any hardware or software.
Is more sensitive and specific to cognitive impairment than other motor assessments.
Is feasible for amnestic Mild Cognitive Impairment (MCI) patients and mild AD.
Can be self-administered at home.
No ceiling effect and can show improvement with practice.

This is in contrast to other more experimental motor measures that have been proposed within AD, which require demanding technology (e.g., movement sensors, motion capture technology, electromyography, or transcranial magnetic stimulation) or do not show strong prognostic effects at baseline (e.g.). The simplified task apparatus (stopwatch, beans, cups, spoon) enable it to be administered in a wide range of settings (e.g., primary care, community health), now including contactless in-home (see above). Furthermore, our work has been scientifically rigorous, doing numerous studies to determine how much the task is subject to practice effects, lateralization effects (i.e., handedness), aging effects, and cognitive effects, as well as testing it against other motor tasks. More details about the motor task itself are provided below in ‘PROCEDURES’.

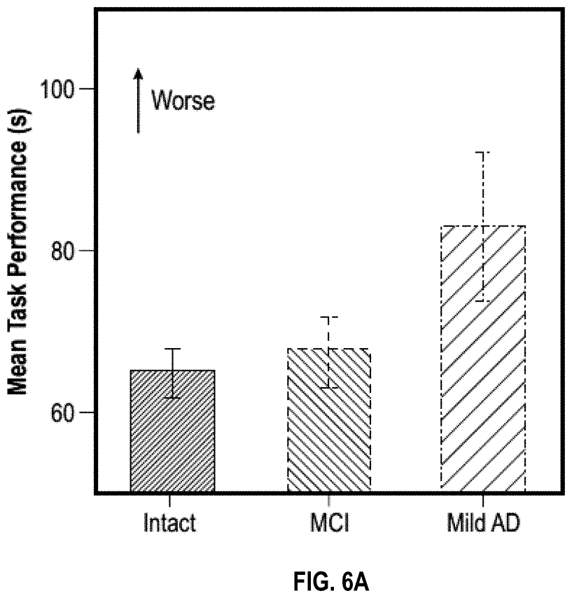

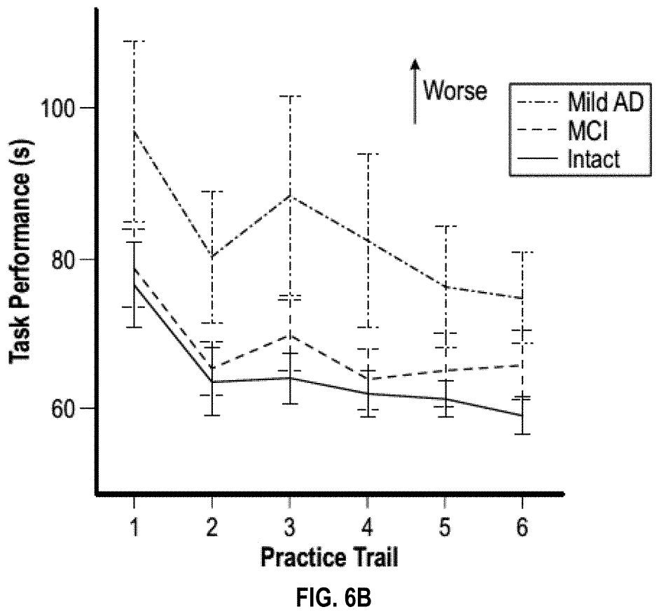

Data from 40 participants (16 cognitively intact, 17 amnestic MCI, and 7 mild AD) from a project within APPE show that the motor task was feasible for all groups, including those diagnosed with mild AD. However, overall task performance was worse (FIG. 6A) and also more variable across trials with more impaired disease status (FIG. 6B), with the cognitively intact group showing stable and improved performance with additional practice. This not only shows that the motor task is feasible for participants with AD, and because it lacks any ceiling effect, our data show that its practice effects track with disease status as well. FIGS. 6A-6B also show the value of looking at performance across multiple trials of the motor task during acquisition (i.e., a performance curve), rather than the “one-and-done” approach that may mask group differences. As noted above, our group has extensively characterized the acquisition and learning of this task across multiple time frames. The use of motor task acquisition to probe motor practice effects in this proposal is highly consistent with the scope of the original APPE study that is investigating the relationship between practice effects on cognitive tests and AD biomarkers.

### Motor Task Acquisition is Related to Amyloid, Hippocampal Volume, and APOE.

Data from a cohort of 36 amnestic MCI participants (ages 65-84) (Duff, R01AG045163) show that adding task performance on a trial later in acquisition (e.g., trial 4) to a multivariate linear regression model improved the variance explained in 18F-Flutemetamol standardized uptake value ratios (SUVR) by over 50% (Adjusted R2 of 0.15 improves to 0.25). This is even after controlling for age, sex, education, delayed memory, and ADL function. In this sample, 26 were amyloid-positive (Aβ+) and 10 were amyloid-negative (Aβ−). The Aβ− group was faster (59.4 seconds; 95% CI [53.9, 64.9]) on the motor task than the Aβ+ group (65.6 seconds; 95% CI [58.4, 72.7]), even though the groups were comparable in delayed memory (˜5th percentile). This further shows that the motor task was more sensitive to amyloid than memory scores. Thus, adding motor task performance to the nominal logistic regression model improved the overall AUC value from 0.84 (good) to 0.90 (excellent), as well as model specificity (50% to 60%), again while controlling for age, sex, education, delayed memory, and ADL function. Model selection criteria also indicated better fits with the addition of the motor task (lower AIC and BIC). A permutation test was run that varied motor task cut-off threshold across the range of performance times observed in this sample, followed by a calculation of the resulting odds ratio for amyloid positivity. The cut-off value with the highest odds ratio was a task performance of 68 seconds acquired by trial 4 with an odds ratio of 4.76. These data show that adding the task as a predictor increases the likelihood of identifying amyloid positivity by nearly 5 times, while costing essentially nothing in terms of dollars and time.

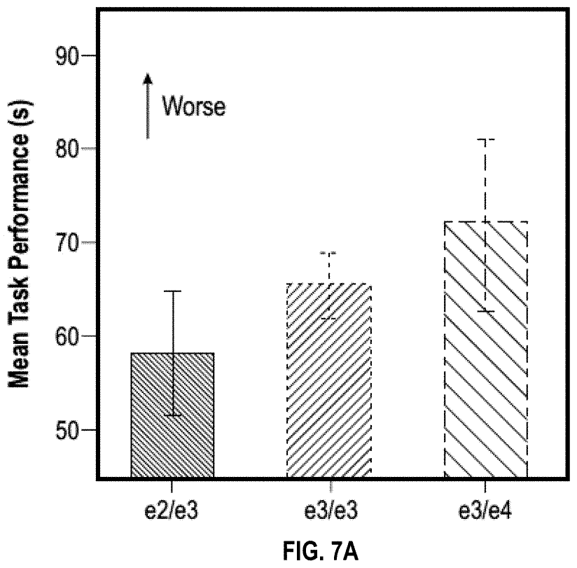

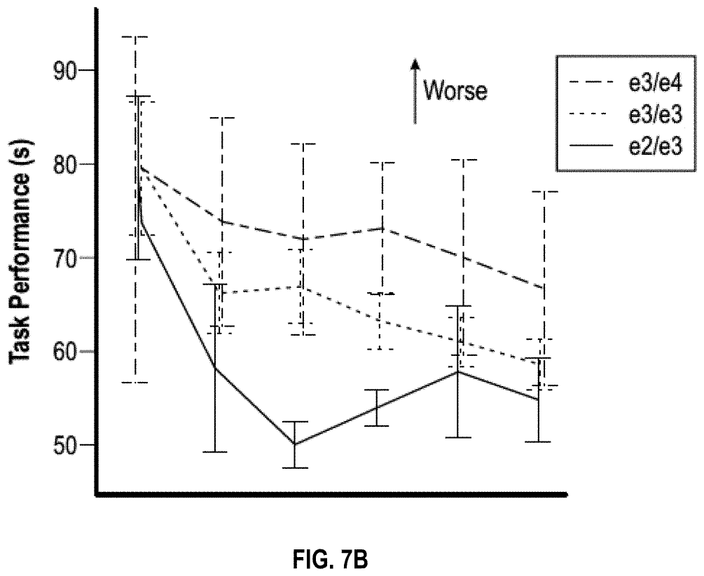

Additional biomarkers such as hippocampal volume and APOE were also collected at baseline in the pilot APPE sample. Regardless of disease status, mean task performance over 6 practice trials was significantly related to bilateral hippocampal volume (measured via MRI and normalized to brain volume) (std β=−0.40; p=0.03), even after controlling for age, education, and delayed memory score (all std β<1.341; all n.s.). This indicated that worse performance on the motor task (slower times) were associated with smaller hippocampal volume, and that motor task performance had the largest effect size compared to other predictors. Similar trends were observed for variability in task performance (standard deviation over 6 trials; p=0.03) as well as performance at the end of practice (p=0.03). (This is consistent with other analyses). Mean task performance also appears to vary with APOE status, as does task acquisition, such that e4 carriers have worse performance across trials than non-e4 carriers; FIG. 7 also shows how e2 carriers are faster (better) compared to other genotypes. To avoid conflating disease status and APOE genotype, only cognitively-intact APPE participants are shown in FIG. 7.

### Motor Task Acquisition can Change Over One Year.

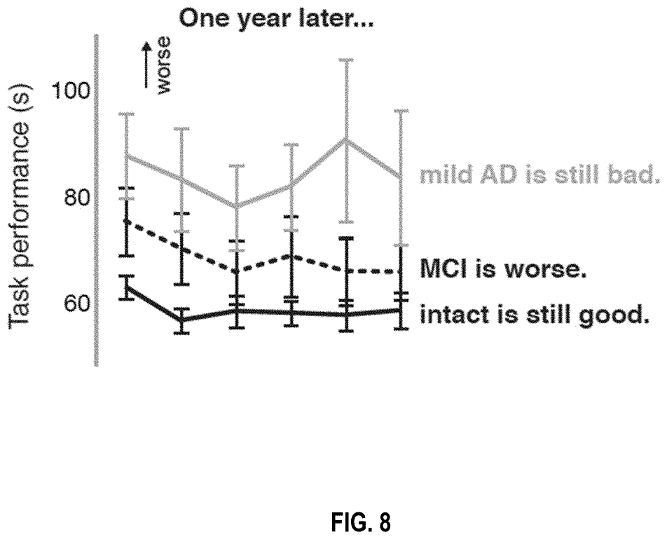

The original APPE project follows individuals over one year, and re-categorizes them as intact, MCI, or AD based on the ADNI criteria. Some participants originally categorized as MCI at baseline did convert to AD (see Aim 3 below). We also collected motor task acquisition data again at one-year follow-up. As shown in FIG. 8, the MCI group's performance across the 6 trials was worse than at baseline (average 73.9 seconds vs. 67.6 seconds), and worse than the intact group. These data show that is feasible to collect motor task acquisition data at multiple time points, and that ‘practice effects’ over multiple trials of the task at a given time point may change with disease progression, especially for higher-risk groups. These data also further validate our approach by showing that acquisition reliably tracks with disease severity.

### Motor Task Acquisition can Predict Disease Conversion.

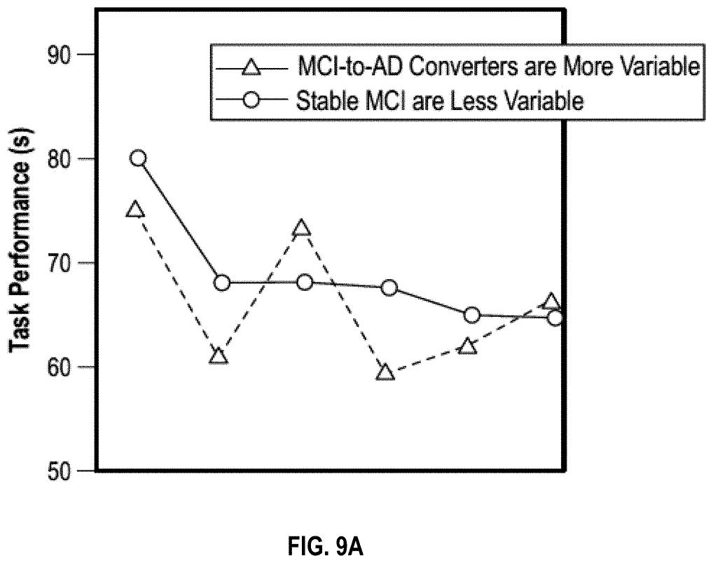

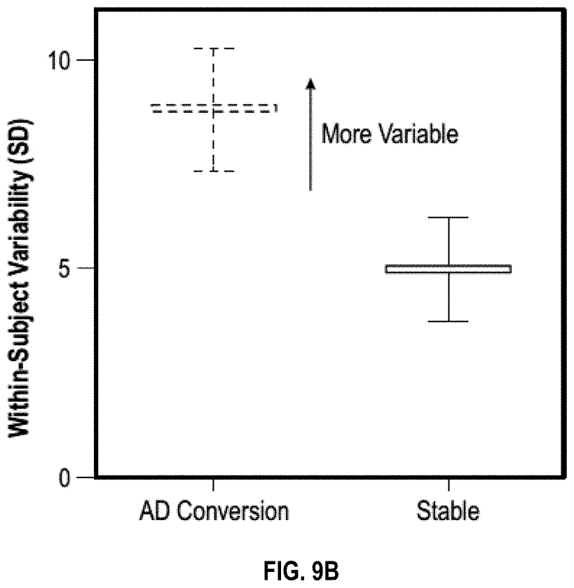

Of the 17 MCI participants within our pilot APPE sample, 9 (56%) converted to AD over one year, as defined by ADNI criteria, while 7 (44%) remained stable. As shown in FIG. 9A, those who converted to AD (gray) were much more variable during acquisition compared to those who were stable (black), particularly after the first trial (as shown in the blue box). As such, the AD conversion group had higher within-subject variability across these trials (FIG. 9B) than the stable group (p=0.07). This is also consistent with another MCI publication, in which motor task performance at the end of acquisition significantly predicted one-year change in both objective and subjective ADL function, even after controlling for baseline ADL function and cognition. Thus, there is strong evidence that our motor paradigm may improve prediction of disease conversion and functional decline.

Proof of coordination across institutions: There is no concern about the quality of the motor data being collected at the APPE cohort site (Univ. of Utah). Dr. Schaefer (MPI: Motor task expertise) has developed a task administration manual and has already traveled to the University of Utah several times to train the APPE staff to administer the motor task. To confirm the reliability of motor task acquisition data collected at the University of Utah, data from the 16 cognitively-intact APPE participants (mean±SD RBANS DMI: 115±11.1) were compared to age- and sex-matched cognitively-intact participants (RBANS DMI: 109±10.1) from an ASU lab, where the motor task was developed and prototyped. None of the practice trials were significantly different based on collection site (all p>0.99), nor a validation trial with the dominant hand (p=1.0). Table 2 shows the comparison between the cohort and development sites across several measures of task performance.

TABLE 2
Comparison of motor task data between
cohort and development sites.

| | | | | Mean task |
|---|---|---|---|---|
| | | Mean task | Mean task | performance |
| | Mean task | acquisition | acquisition | w/dominant |
| | performance | (SD) | (last trial) | hand |
| cohort site (Utah) | 66.53 s | 8.98 s | 62.50 s | 47.83 s |
| Development site | 65.24 s | 8.94 s | 63.75 s | 46.78 s |
| (ASU) | | | | |

Note that the dominant hand (regardless of site) is much faster than the nondominant hand providing evidence of why the nondominant hand is used to investigate practice effects (i.e., no ceiling effect). These data clearly show the ability of the APPE staff to reliably and independently collect motor task acquisition.

Current Status

In this study, the Enriching Clinical Trials Requiring Amyloid Positivity with Practice Effects (APPE) study, we have enrolled 151 participants into the study. Using results from our cognitive assessment at Visit 1 and the ADNI-based criteria from our research protocol, 68 were classified as cognitively intact, 36 as amnestic MCI (single- or multi-domain), and 47 as mild Alzheimer's disease. 130 of them have completed 1) MRIs of the brain for volumetric, diffusion tensor imaging, and functional connectivity analysis, 2) blood draws for APOE testing, and 3) amyloid PET scans using Flutemetamol. This demonstrates that we have a sufficient sample with banked data. (see ‘PARTICIPANTS’ below). 86 participants completed their one-year follow-up visit, in which we repeat cognitive testing and reclassify participants according to ADNI-based criteria to look for instances of conversion or reversion. A subset of these individuals (n=40) was involved in a pilot project that included collecting motor task acquisition data at baseline and one-year follow-up, providing the data shown above.

Participants

The study leverages banked data from at least 108 older adults (65 years or older) that fall into one of three categories: probable AD dementia, amnestic MCI, or cognitively intact. We elected to include participants from three groups (AD, MCI, and intact) to increase the variability of cognitive scores and biomarker results, and most accurately perform studies. Preliminary data suggest differences in motor task acquisition between these three groups. For example, progressively greater amounts of amyloid positivity from intact to MCI to AD, as well as variability within groups, have been reported, which is consistent with our preliminary motor data. Probable AD dementia will be diagnosed according to the NIA-AA criteria (e.g., insidious onset, worsening of cognition, amnestic or non-amnestic presentation, no other causative conditions). Amnestic MCI will be diagnosed according to the NIA-AA core clinical criteria (e.g., concern of change in cognition, impairment in one or more cognitive domains, preservation of functional abilities, not demented). To increase the likelihood that these participants reflect MCI due to AD, memory must be one of the cognitive domains that are impaired. Cognitively intact participants will not meet criteria for MCI or dementia. To further operationally define our groups and increase generalizability to existing literature, inclusion criteria will follow:

ADNI protocol (see below).

### Exclusion Criteria:

History of major stroke, head injury with loss of consciousness of >30 minutes, or other neurological or systemic illness that may affect cognition
Current or past major psychiatric illness (e.g., schizophrenia, bipolar affective disorder)
History of substance abuse
Current use of antipsychotics or anticonvulsant medications
Inadequate vision, hearing, and manual dexterity to participate in the cognitive assessments

Cholinesterase inhibitors and other cognitive enhancing medications will be allowed, but their type and dosages will be recorded at each visit, coded, and considered in statistical analyses. Similarly, other common medical comorbidities (e.g., diabetes, hypertension) will be recorded at each visit, coded, and considered as potential covariates. Including these medical and medication comorbidities should make findings more generalizable for future applications. Inclusion and exclusion criteria will be determined via self-report, review of medical records, objective testing, and interview with participant and a knowledgeable informant.

### Procedure:

After informed consent/assent of participants and informants, baseline and one-year follow-up visits will occur.

Baseline visit: Cognitive assessment+Motor task acquisition. (˜120 minutes) Following a brief interview to confirm demographic information, cognitive and daily functioning (measured with the Repeatable Battery for the Assessment of Neuropsychological Status [RBANS] and the Alzheimer's Disease Cooperative Study Activities of Daily Living scale [ADCS-ADL]), medical and psychiatric history, participants will complete the measures in Table 3 to classify their diagnostic group. These measures and cutoffs were chosen based on ADNI to make our results more generalizable to the existing literature.

TABLE 3
Diagnostic classification battery based on ADNi criteria.

| Measure | Cutoff for intact | Cutoff for MCI | Cutoff for AD |
|---|---|---|---|
| MMSE | 24-30 | 24-30 | 20-26 |
| WMS-R Logical Memory | ≥9 if educ ≥16 years | ≤8 if educ ≥16 years | ≤8 if educ ≥16 years |
| II (Paragraph A only) | ≥5 if educ 8-15 years | ≤4 if educ 8-15 years | ≤4 if educ 8-15 years |
| | ≥3 if educ 0-7 years | ≤2 if educ 0-7 years | ≤2 if educ 0-7 years |
| CDR (Overall) | 0 | 0.5 | 0.5 or 1 |
| GDS (15-item version) | <6 | <6 | <6 |

Participants will also complete 6 consecutive trials of the motor task, which will comprise task acquisition. We use the term “task acquisition” here because it refers to the initial practice of a motor skill. We avoid using the term “motor learning”, as that is characterized a relatively permanent change in performance due to extensive practice. Instead, we will leverage the susceptibility of our motor task to change due to repeated exposure (i.e., a practice effect), which aligns with the original scope of the APPE study. As such, a 6-trial exposure to the motor task allows us to generate performance curves, as shown in ‘DATA’. For transparency and dissemination, a full visual description of the timed motor task is freely available online at Open Science Framework. To summarize, participants use a standard plastic spoon to acquire two raw kidney beans at a time from a central cup to one of three distal cups arranged at a radius of 16 cm at 40°, 0°, and 40° relative to the central cup. All cups are the same size (a single-serving Greek yogurt cup, 9.5 cm diameter and 5.8 cm deep) and are secured to a board. Participants acquire and transport two beans at a time to transport to the ipsilateral cup, then the middle cup, then the contralateral cup, and then repeat this sequence four more times for a total of 15 out-and-back movements. This equals one trial. Participants will be tested using their nondominant hand. The nondominant hand is used (rather than the dominant hand) to minimize ceiling effects and allow the possibility of improvement with practice. Recent work also shows complex movements in the nondominant hand of older adults may be more sensitive to white matter hyperintensities than the dominant hand, or simple movements (i.e., finger tapping) with either hand.

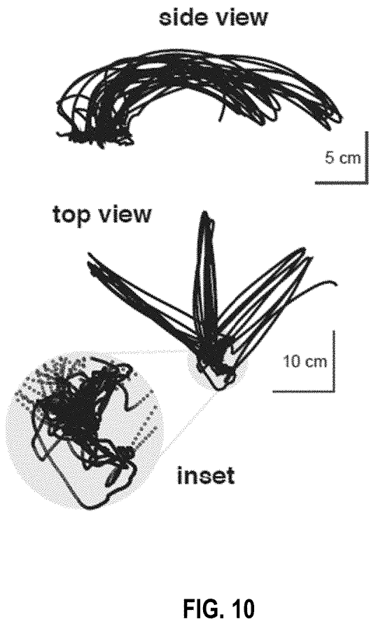

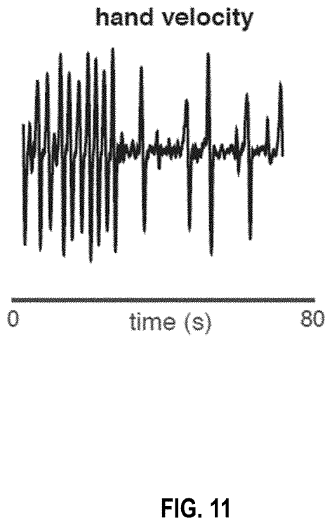

Participants are instructed to move ‘as quickly and accurately as possible’. Task performance is measured as trial time (in seconds), i.e., how long it took to complete 15 movements, such that lower values indicate better performance. One measure of motor task acquisition will be the change in performance from trial 1 to trial 6 (i.e., a practice effect). Additional measures of within-subject variability (standard deviation) and overall average performance (mean) will also be calculated. This approach is highly consistent with the scope of the original APPE study in investigating the relationship between practice effects on cognitive tests and AD biomarkers. Movement errors, such as dropping beans mid-reach, will also be recorded; for reference, ˜1 error/participant was made in the pilot APPE project across all 6 trials, with no significant difference between intact, MCI, or AD groups (p=0.50). Nevertheless, errors will be recorded and analyzed as a tertiary outcome. FIG. 10 shows a side and top view of the hand's movement over the course of one trial, as well as the hand's velocity (FIG. 11) We have collected detailed kinematic data of the hand, including in older adults. Using kinematic data, we have shown that the fine motor aspect of the task (i.e., loading beans two-at-a-time onto the spoon, shown in the figure inset) is sensitive to aging and cognition, whereas the reaching phase is not. Furthermore, we have shown that the integrity of frontoparietal white matter tracts is related to task acquisition, and our behavioral data suggests that this task taps into visuospatial memory mediated by the hippocampus. Thus, our experimental research has and continues to investigate which aspects of this motor task are the ‘active ingredients’ most sensitive and specific to AD progression. Without being bound by theory, these data will provide data in cognitively intact and impaired individuals that will inform the underlying mechanisms of this task as an enrichment and/or prognostic tool in the future.

### One-Year Follow-Up Visit: Cognitive Assessment+Motor Task Acquisition. (˜120 Minutes)

Approximately 1 year later, participants will return to repeat the procedures in their baseline visit. In addition to re-classifying participants according to the ADNI criteria, these measures will be compared to those obtained at the Baseline assessment to examine how motor task acquisition is related to AD progression over time and characterize change in task acquisition itself over time.

Banked data from APPE. The following data are collected in all APPE participants via amyloid-PET imaging, MRI imaging, and APOE genotyping. Extensive neuropsychological data and daily functioning data are also collected. All [18F]Flutemetamol PET images are sent to the GE HealthCare AW workstations for review and post processing with CortexID Suite. PET and MR images are sent to our research PACS system for archiving. All measures are banked in REDCap and will be compared with motor task acquisition data in this project.

Amyloid-PET imaging: 18F-39-F-6-OH-BTA1 (18FGE067) known as Flutemetamol, a structural thioflavin analog of PIB, has been examined as a tracer for brain amyloid. This is the tracer used in the ongoing APPE study. It behaves similar to Pittsburgh Compound B (PIB), but has a shorter half-life (110 vs. 20 minutes), making it more practical for research and clinical use. Cortical composite retention ratios between Flutemetamol and PIB in MCI or AD have been shown to correlate at 0.88-0.99. Flutemetamol was approved by the FDA on 10/23/15 (Vizamyl) as a radioactive diagnostic agent indicated for PET imaging to estimate β-amyloid neuritic plaque density in patients with cognitive impairment being evaluated for AD or other causes of cognitive decline (General Electric Healthcare).

The Flutemetamol is prepared under IND #109,760 (Hoffman IND Holder), and administered as a dose of 5.0 mCi via IV pushed over about 30-60 seconds, followed with an intravenous flush of 5-15 mL of 0.9% sterile sodium chloride injection. A helical CT scan (via GE DST PET/CT) is performed over the same anatomic range corresponding to the PET scan for attenuation correction. The PET emission scan starts immediately after the CT scan (120 kVp, 0.5 s rotation speed, 50 mA tube current, 8×1.25 mm collimation, and a pitch of 1.35). The PET emission study is performed for 20 minutes in 3D mode with CT reconstruction parameters as follows: 128×128 matrix; FORE-Iterative; Subsets, 30; Iterations: 5; No Z Axis filter; Loop filter 2.00; and Recon Diameter: 25.6 cm. Primary analysis of [18F]Flutemetamol binding occurs using a regional semi-quantitative technique, described by and refined by. The primary measure is a global composite of standardized uptake value ratios (SUVRs) in the cerebral cortex, obtained and normalized to the pons. The following regions are averaged for the global composite: lateral frontal, lateral temporal, lateral parietal, anterior cingulate, and posterior cingulate. CortexID is used to calculate regional and global composite SUVRs, with a positivity threshold of 0.62. Secondary assessment of amyloid plaque burden uses visual assessment of abnormal cortical [18F]Flutemetamol uptake as outlined in the prescribing information of Vizamyl. This binary assessment labels images as positive (i.e., increased/abnormal Flutemetamol uptake) or negative.

Magnetic Resonance Imaging: Structural imaging is performed using a sagittal MP2RAGE pulse-sequence to obtain high quality whole-brain 1 mm isotropic T1w images with improved signal homogeneity in ˜7 minutes. Diffusion tensor imaging is performed using a diffusion-weighted, multi-band EPI sequence with a slice celeration factor of 3, whole brain coverage with 1.5 mm isotropic resolution. Resting functional BOLD MRI (fMRI) is performed using a T2*-weighted multiecho, multiband fMRI sequence (TR=1550 ms for 3 volumes a 3 TE levels) for 30 minutes aggregate scan time in 2 15-minute scans at 2 mm isotropic spatial resolution. The primary MRI measure is hippocampal atrophy, one of the most widely used AD biomarkers. It has been linked to disease stage and progression. ADNI and other studies have shown the value of this biomarker in clinical trials and its relationship to other biomarkers of AD. It is measured through structural MRI scans that are processed using FreeSurfer image analysis suite. Thickness and subcortical volume measurements are extracted via image processing using the longitudinal processing stream. Other banked MRI measures include variables from diffusion tensor imaging (DTI), whole brain white matter-tractography, structural connectivity, and multiband/multiecho BOLD functional connectivity, which can be included in exploratory analyses with motor task acquisition across the sample as well.

APOE genotyping: When the IV line is started for the [18F]Flutemetamol injection, 3 mL of whole blood are collected in a 10 cc lavender top tube, which is gently mixed to assure combination with EDTA anticoagulant. The sample remains stable at ambient temperature for up to 72 hours. It is delivered to the ARUP Laboratories, who conduct Polymerase Chain Reaction and Fluorescence Monitoring using hybridization probes for APOE genotyping. Results are reported in <7 days, and are not shared with participants.

Cognitive and functional measures: In addition to ADNI classification battery, APPE has collected multiple cognitive measures on its participants, including the Repeatable Battery for the Assessment of Neuropsychological Status (RBANS), Hopkins Verbal Learning Test-Revised (HVLT-R), Brief Visuospatial Memory Test-Revised (BVMT-R), Symbol Digit Modalities Test (SDMT), Trail Making Test (TMT), and the Reading subtest of the Wide Range Achievement Test-IV (WRAT-IV). The Quick Dementia Rating System (QDRS) is also collected on participants using collateral input to estimate a CDR score, which is the most widely-used measure assessing cognition and functioning in AD. All of these measures are collected at a baseline visit, a subset is collected at a one-week visit (HVLT-R, BVMT-R, SDMT, TMT), and all are collected at a one-year visit.

### Consideration of Biological and Psychosocial Variables

Sex differences. Population-based studies have reported no sex differences in amyloid positivity or other biomarkers. Similarly, our own data have shown no sex differences on these variables (amyloid, p=0.74; hippocampal volume, p=0.48; APOE genotype, c2 test p=0.22), nor in motor task acquisition (average performance, p=0.15; variability, p=0.13; last trial, p=0.16). This is actually an advantage of our task relative to other current motor assessments, such as grip strength or Purdue Pegboard, since these both consistently show sizeable sex differences even after controlling for age. However, since we will be recruiting both males and females, we will use sex as a covariate to test for any differences in our Aims. The APPE cohort is ideal for studying sex differences as well due its relatively balanced sex distribution, with 57% female and 43% male.

Depressive symptom differences. Given that the motor task's primary outcome variable is timed (i.e., how quickly participants perform the task), we acknowledge that the presence of depressive symptoms could influence task performance. Higher levels of self-reported depressive symptoms have been associated with slower gait speeds, which might suggest a similar relationship with upper extremity movement speeds. We do not, however, see this in our preliminary data, as motor task variability and performance on the last trial were unrelated to Geriatric Depression Scale (all p>0.28), even when controlling for age, sex, dementia diagnosis, and marital status. In a previous study, we also showed that baseline depressive symptoms (measured with the Center for Epidemiologic Studies—Depression scale) were unrelated to fine motor skill, further suggesting that upper extremity movements that involve a fine motor component may be less susceptible to psychosocial factors than lower extremity movements like gait and balance. Nevertheless, the extent to which a participant has any depressive symptoms could still mediate some of this proposal's main effects, and the ongoing collection of depressive symptom data within APPE will allow for secondary analyses.

Computing Device: Referring to FIG. 13, an example computing device 1200 including a processor is illustrated which may take the place of the processor 104 and be configured, via one or more of an application 1211 or computer-executable instructions, to execute functionality described herein. More particularly, in some embodiments, aspects of the predictive methods herein may be translated to software or machine-level code, which may be installed to and/or executed by the computing device 1200 such that the computing device 1200 is configured to execute functionality described herein. It is contemplated that the computing device 1200 may include any number of devices, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments, and the like.

The computing device 1200 may include various hardware components, such as a processor 1202, a main memory 1204 (e.g., a system memory), and a system bus 1201 that couples various components of the computing device 1200 to the processor 1202. The system bus 1201 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing device 1200 may further include a variety of memory devices and computer-readable media 1207 that includes removable/non-removable media and volatile/nonvolatile media and/or tangible media, but excludes transitory propagated signals. Computer-readable media 1207 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computing device 1200. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 1204 includes computer storage media in the form of volatile/nonvolatile memory such as read only memory (ROM) and random access memory (RAM). A basic input/output system (BIOS), containing the basic routines that help to transfer information between elements within the computing device 1200 (e.g., during start-up) is typically stored in ROM. RAM typically contains data and/or program modules that are immediately accessible to and/or presently being operated on by processor 1202. Further, data storage 1206 in the form of Read-Only Memory (ROM) or otherwise may store an operating system, application programs, and other program modules and program data.

The data storage 1206 may also include other removable/non-removable, volatile/nonvolatile computer storage media. For example, the data storage 1206 may be: a hard disk drive that reads from or writes to non-removable, nonvolatile magnetic media; a magnetic disk drive that reads from or writes to a removable, nonvolatile magnetic disk; a solid-state drive; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules, and other data for the computing device 1200.

A user may enter commands and information through a user interface 1240 (displayed via a monitor 1260) by engaging input devices 1245 such as a tablet, electronic digitizer, a microphone, keyboard, and/or pointing device, commonly referred to as mouse, trackball or touch pad. Other input devices 1245 may include a joystick, game pad, satellite dish, scanner, or the like. Additionally, voice inputs, gesture inputs (e.g., via hands or fingers), or other natural user input methods may also be used with the appropriate input devices, such as a microphone, camera, tablet, touch pad, glove, or other sensor. These and other input devices 1245 are in operative connection to the processor 1202 and may be coupled to the system bus 1201, but may be connected by other interface and bus structures, such as a parallel port, game port or a universal serial bus (USB). The monitor 1260 or other type of display device may also be connected to the system bus 1201. The monitor 1260 may also be integrated with a touch-screen panel or the like.

The computing device 1200 may be implemented in a networked or cloud-computing environment using logical connections of a network interface 1203 to one or more remote devices, such as a remote computer. The remote computer may be a personal computer, a server, a router, a network PC, a peer device or other common network node, and typically includes many or all of the elements described above relative to the computing device 1200. The logical connection may include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computing device 1200 may be connected to a public and/or private network through the network interface 1203. In such embodiments, a modem or other means for establishing communications over the network is connected to the system bus 1201 via the network interface 1203 or other appropriate mechanism. A wireless networking component including an interface and antenna may be coupled through a suitable device such as an access point or peer computer to a network. In a networked environment, program modules depicted relative to the computing device 1200, or portions thereof, may be stored in the remote memory storage device.

Certain embodiments are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure the processor 1202, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

Computing systems or devices referenced herein may include desktop computers, laptops, tablets e-readers, personal digital assistants, smartphones, gaming devices, servers, and the like. The computing devices may access computer-readable media that include computer-readable storage media and data transmission media. In some embodiments, the computer-readable storage media are tangible storage devices that do not include a transitory propagating signal. Examples include memory such as primary memory, cache memory, and secondary memory (e.g., DVD) and other storage devices. The computer-readable storage media may have instructions recorded on them or may be encoded with computer-executable instructions or logic that implements aspects of the functionality described herein. The data transmission media may be used for transmitting data via transitory, propagating signals or carrier waves (e.g., electromagnetism) via a wired or wireless connection.

It should be understood from the foregoing that, while particular embodiments have been illustrated and described, various modifications can be made thereto without departing from the spirit and scope of the inventive concept as will be apparent to those skilled in the art. Such changes and modifications are within the scope and teachings of this inventive concept as defined in the claims appended hereto.

What is claimed is:

1. A system for prognosis of early cognitive decline or other neurological concerns, comprising:
a plurality of testing components configured for interaction with an individual to execute one or more motor tasks, the one or more motor tasks including engagement of upper extremities of the individual to acquire at least one object of the plurality of testing components from a source location and transport the one or more objects to a target location; and
a processor configured to:
access a plurality of trial times and test scores associated with the one or more motor tasks, and
compute a motor test score, the motor score reflecting a potential concern for a neurological condition based upon a predefined score threshold.

2. The system of claim 1, wherein the one or more motor tasks includes a plurality of goal directed movements to visible target locations, spatially arranged such that at least one target is located ipsilateral to the reaching extremity, at least one target is located contralateral to a reaching extremity, and one target is located along the individual's midline.

3. The system of claim 1, wherein the one or more motor tasks includes a sequence of target locations to indicate an order in which the individual must transport the one or more objects, the sequence of target locations being the same across a plurality of trials of the one or more motor tasks.

4. The system of claim 1, further comprising:
a user interface executed by a computing device, the computing device configured to provide, via the user interface, a stopwatch function and a scoring function that the individual engages to accommodate aggregation of the plurality of trial times and test scores for access by the processor.

5. The system of claim 1, wherein the plurality of testing components includes:
a tool that the individual engages to move the one or more objects as part of the one or more motor tasks.

6. The system of claim 5, wherein the tool includes a handle and a repository, the repository configured to receive the one or more objects as part of the one or more motor tasks.

7. The system of claim 5, wherein the tool includes a sensor configured to measure changes in a grip force of the individual during the one or more motor tasks.

8. The system of claim 5, wherein the tool includes a sensor configured to measure electrodermal response due to physiological arousal.

9. The system of claim 5, wherein the one or more motor tasks includes a sequence of a plurality of movements of the one or more objects by the individual using the tool defining a trial.

10. The system of claim 9, wherein the processor derives the motor score from trial time and test score data associated with multiple instances of the trial, higher values being associated with a neurological concern.

11. The system of claim 1, wherein the plurality of testing components include are disposable and packaged within a kit configured for portable and efficient testing.

12. The system of claim 1, wherein the motor score reflects decreased motor task acquisition including variability across trials of the one or more motor tasks or lack of improvement of the one or more motor tasks with practice.

13. The system of claim 1, wherein to compute the motor test score the processor computes a standard deviation of the plurality of test scores over a predetermined time period.

14. A method for non-invasive prognosis of early cognitive decline or other neurological concerns, comprising:
providing a plurality of test elements, the plurality of test elements configured for interaction with an individual to execute a plurality of trials of a motor task according to a predetermined sequence, the motor task including engagement of upper extremities of the individual to acquire a first object of the plurality of test elements from a source location and transport the first object to a first target location; and
executing, by a processor, steps of:
accessing test data including a first plurality of trial times associated with the plurality of trials of the motor task, and
computing a motor test score, the motor score reflecting a potential concern for a neurological condition based upon a predefined score threshold.

15. The method of claim 14, further comprising providing the plurality of test elements via a kit, the plurality of test elements being disposable.

16. The method of claim 14, further comprising accessing, by the processor, the test data via a computing device executing a user interface configured for tracking trial times associated with execution of the motor task.

17. The method of claim 14, wherein the predetermined sequence includes goal-directed movements of a plurality of objects from a source receptacle to one or more target receptacles.

18. The method of claim 14, further comprising:
executing, by the processor, steps of:
accessing a second plurality of trial times associated with a second plurality of trials of the motor task executed by the individual,
computing a subsequent motor test score, and
determining a delta between the motor test score and the subsequent motor test score to assess potential neurological concern.

19. A kit for prognosis of early cognitive decline or other neurological concerns, comprising:
a container;
a plurality of testing elements including one or more objects and a plurality of receptacles, the container configured for secure storage and transport of the plurality of testing elements; and
an instruction for guiding an individual to complete one or more motor tasks using the plurality of testing elements according to a predetermined sequence configured for detecting a neurological concern.

20. The kit of claim 19, further comprising a tool configured for storage within the container that accommodates goal-directed movements of the one or more objects defined by the sequence.
